(19) United States
(12) Patent Application Publication
(10) Pub. No.: US 2015/0110738 A1
(43) Pub. Date: Apr. 23, 2015

(54) METHODS AND COMPOSITIONS FOR GENERATING AND USING ALLOGENEIC SUPPRESSOR CELLS

(71) Applicant: University of Southern California, Los Angeles, CA (US)

(72) Inventor: David Horwitz, Santa Monica, CA (US)

(21) Appl. No.: 14/398,827

(22) PCT Filed: May 2, 2013
(86) PCT No.: PCT/US2013/039287

§ 371 Date: Nov. 4, 2014

Related U.S. Application Data
(60) Provisional application No. 61/798,530, filed on Mar. 15, 2013.
(60) Provisional application No. 61/642,948, filed on May 4, 2012.

(51) Int. Cl. A61K 35/17; A61K 38/20; C12N 5/0783; A61K 39/395

(52) U.S. Cl. CPC A61K 35/17; A61K 39/3955; A61K 38/2013; C12N 5/0637; A61K 2035/122
USPC 424/85.2; 424/93.71; 435/377; 435/325; 435/374

(57) ABSTRACT

The present invention is directed to generating suppressor cells by treating naive T cells with a suppressor-inducing composition such as anti-CD3, anti-CD28, IL-2, TGF-β, or some combination thereof. Such suppressor cells are administered to patients to prevent or treat immune disorders and are allogeneic to the patient.

### RELATED APPLICATIONS

This application claims the benefit of U.S. Provisional Application Ser. No. 61/798,530, filed Mar. 15, 2013, and U.S. Provisional Application Ser. No. 61/642,948, filed May 4, 2012 under 35 U.S.C. 119(e) are herein incorporated in its entirety by reference.

### TECHNICAL FIELD

The present invention is directed to generating suppressor cells by treating naïve T cells with a suppressor-inducing composition such as anti-CD3, anti-CD28, IL-2, TGF-β, or some combination thereof. Such suppressor cells are administered to patients to prevent or treat immune disorders and are allogeneic to the patient.

### BACKGROUND OF THE INVENTION

Since regulatory T cells (Tregs) control pathogenic self-reactive cells, they have therapeutic potential for autoimmune diseases. Some clinical trials have utilized Tregs isolated from blood that are then expanded to large numbers, and transferred to the patient. Alternatively, Tregs that have been induced ex-vivo from conventional T cells have also been candidates for clinical trials. Attention is often focused on CD4+CD25+ Foxp3+ cells due to their role in preventing autoimmunity. However, these Tregs are difficult to expand from the small numbers that can generally be isolated, and their functional properties decrease after expansion. Moreover, the pathogenic memory T cells which are predominant in established autoimmune diseases appear to be resistant to suppression by CD4 Tregs. An alternative to CD4 Tregs are CD8⁺ suppressor cells.

Antigen-specific CD8⁺ suppressor cells generated in a mixed lymphocyte reaction have the ability to prolong cardiac allografts. CD8⁺ cells treated with TGF-β develop suppressive activity and TCR transgenic CD8⁺ cells treated with TGF-β become Foxp3+ and develop potent suppressive activity that can be distinguished from their cytolytic effects

One challenge with using CD4+ and CD8⁺ suppressor cells for treatment of immune disorders is that the patient's immune system will act to reject allogeneic suppressor cells unless immunosuppressive therapies (which often have their own unwanted side effects) are administered. There remains a need for therapies that can utilize allogeneic suppressor cells without need of further immunosuppressive therapies to prevent rejection.

### SUMMARY OF THE INVENTION

Accordingly, the present invention provides compositions and methods for generating suppressor cells and for treating immune disorders in a patient by administering allogeneic suppressor cells to that patient. In certain embodiments, the allogeneic suppressor cells administered to the patient are activated CD8⁺ cells. In a preferred embodiment, suppressor cells, such as CD8⁺ suppressor cells, are administered to treat or prevent a disease other than graft vs. host disease. In other embodiments the allogeneic suppressor cells are administered to treat an immune disorder such as an autoimmune disease. In yet other embodiments the suppressor cells are used to treat or prevent tissue (e.g. skin grafts) or organ rejection.

In further aspects and in accordance with the above, the present invention provides methods in which the suppressor cells used to treat a patient are generated by contacting blood, a component of blood comprising naïve CD8⁺ cells or isolated naïve CD8⁺ cells (which are allogeneic to the patient) with a suppressor-inducing composition which includes one or more mitogens and cytokines (e.g. anti CD-28 antibodies, anti-CD3 antibodies and IL-2) to activate the naïve CD8⁺ cells to become suppressor cells (sometimes referred to as activated CD8⁺ Tregs, also referred to as activated CD8⁺ suppressor cells). In certain embodiments, the anti-CD3 and anti-CD28 antibodies (also referred to herein as “anti-CD3” and “anti-CD28”) are applied using anti-CD3/anti-CD28 coated beads. In some embodiments (and in accordance with any of the above) the CD8⁺ cells are further treated with TGF-β to generate suppressor cells. The suppressor cells can then be expanded prior to administration to the patient.

In a related embodiment, after generation of the allogeneic activated suppressor cells the cells are expanded in the presence of (1) the patient's MHC antigens (HLAs in humans) e.g. for treatment of an autoimmune disease or (2) the patient's MHC antigens and the MHC antigens from the donor of cells, tissue or organs to be transplanted to the patient. Peripheral blood mononuclear cells (PBMCs) can be used for this purpose. Prior to expansion, the cell population of activated cells contains broadly reactive polyclonal CD8⁺ Tregs. To survive these Tregs require continuous antigen stimulation. Therefore, exposure of the CD8⁺ Tregs to PBMCs of the patient and/or donor will select out those CD8⁺ cells that will preferentially expand following introduction into the patient. Such expansion concentrates the CD8⁺ Tregs that are specific for the patient's and/or donor's MHC I antigens. In some embodiments, the patient's PBMCs may be activated and then irradiated to enhance expansion of the CD8⁺ Tregs. In other embodiments IL-2 is also added to enhance CD8⁺ Treg expansion. These MHC antigen stimulated cells are sometimes referred to as expanded suppressor cells or expanded CD8⁺ suppressor cells.

When the patient is receiving allogeneic tissue (e.g. skin), organ or bone marrow from a donor, it is preferred that the CD8⁺ Tregs be derived from the donor or a partially matched third party and expanded in the presence of MHC antigens from the patient. When CD8⁺ Tregs from the donor are used it is preferred that MHC antigens from the patient be used to expand the CD8⁺ Tregs. When partially matched CD8⁺ Tregs are used, in the case of tissue or organ transplantation, the partially matched third party CD8⁺ Tregs are preferably expanded with MHC antigens from the patient and optionally from the donor as well. In the case of bone marrow transplantation or the treatment of graft vs. host disease the partially matched third party CD8⁺ Tregs are preferably expanded with MHC antigens from the donor and optionally with MHC antigens from the patient.

In certain embodiments and in accordance with any of the above, allogeneic suppressor cells used in accordance with the present invention are resistant to rejection by the patient's immune system.

In further embodiments and in accordance with any of the above, suppressor cells of the invention inhibit allogeneic T cells in the patient.

In still further embodiments and in accordance with any of the above, the immune disorder treated with suppressor cells of the invention is an autoimmune disorder, graft versus host disease, or a disorder arising from an individual receiving a foreign tissue or organ allograft.

In yet further embodiments and in accordance with any of the above, suppressor cells of the invention maintain suppressive activity in the presence of IL-2.

In further embodiments and in accordance with any of the above, suppressor cells of the invention express one of or both of TNFR2 and PD-L1.

In further embodiments and in accordance with any of the above, the suppressor cells of the invention are generated from cells obtained from a donor who matches the patient for at least one set of MHC antigens, but does not match all MHC antigens of the patient receiving the suppressor cells.

In still further embodiments and in accordance with any of the above, the allogeneic suppressor cells are administered to the patient without other immunosuppressive therapies.

In still further embodiments and in accordance with any of the above, suppressor cells administered to the patient may be contained in a blood product that is storage stable for a predetermined period of time.

In yet further embodiments and in accordance with any of the above, the suppressor cells are contained in a first blood product selected from a group of blood products, where at least two and up to a thousand or more of the blood products in the group include CD8+ suppressor cells from different individuals that have different MHC antigens (HLAs in humans).

In further embodiments, the suppressor cells are further expanded by culturing in the presence of CD4+ regulatory cells obtained from the patient who will receive the suppressor cells prior to treating that patient with the suppressor cells.

In some aspects and in accordance with any of the above, the present invention provides methods for generating suppressor cells that include the steps of (a) providing naïve CD8⁺ cells, and (b) activating the CD8⁺ cells with a suppressor-inducing composition, such as anti-CD3, anti-CD28 and IL-2, to generate the activated suppressor cells.

In another aspect the invention provides methods for generating expanded CD8+ suppressor cells that include the steps of (a) activating naïve CD8⁺ cells by culturing them in the presence of a suppressor-inducing composition such as anti-CD3, anti-CD28 and IL-2, and (b) expanding the activated CD8⁺ cells in the presence of at least two MHC antigens which are different from each other and from the MHC antigens of activated CD8⁺ cells.

In further aspects and in accordance with any of the above, prior to the activating step, CD8⁺ cells are isolated from blood.

In some embodiments and in accordance with any of the above, the CD8⁺ cells are further treated with TGF-β to generate suppressor cells.

In further embodiments and in accordance with any of the above, suppressor cells generated in accordance with the invention are cultured for a predetermined period of time to expand the suppressor cells. In still further embodiments, the suppressor cells are CD8⁺ cells that are cultured in the presence of allogeneic CD4+ regulatory cells.

In yet further embodiments and in accordance with any of the above, suppressor cells of the invention are stable for storage in a blood bank.

In still further embodiments, and in accordance with any of the above, the suppressor cells of the invention target allogeneic cells when said suppressor cells are administered to a patient.

In some aspects and in accordance with any of the above, the present invention provides a blood product that comprises CD8⁺ suppressor cells that are generated by treating blood or a component of blood comprising naïve CD8⁺ cells with a suppressor-inducing composition (e.g. anti-CD3, anti-CD28 and IL-2). In further embodiments, the blood product is capable of storage in a blood bank.

In further embodiments and in accordance with any of the above, CD8⁺ suppressor cells in blood products of the invention inhibit allogeneic cells in a patient when that blood product is administered to a patient.

In still further embodiments and in accordance with any of the above, subsequent to treating with anti-CD3, anti-CD28 and IL-2, the blood is further mixed with a blood bank storage quantity of anti-coagulant acid-citrate-dextrose solution. In yet further embodiments, the blood is further treated with TGF-β.

In other embodiments, the invention provides a composition comprising expanded CD8⁺ suppressor cells generated by activating naïve CD8⁺ cells by treating blood, a component of blood comprising naïve CD8⁺ cells or isolated naïve CD8⁺ cells with a suppressor-inducing composition to activate the cells and expanding the activated CD8⁺ cells by culturing in the presence of at least first and second different MHC antigens which are allogeneic to the MHC antigens of the CD8⁺ cells. In some embodiments, the first different MHC antigens are from a cell, tissue or organ of a donor and the second different MHC antigens are from a recipient of the cell, tissue or organ.

In some aspects and in accordance with any of the above, the present invention provides a suppressor cell bank that includes a collection of containers where each container contains CD8⁺ suppressor cells from a different individual. In certain embodiments, at least two of said containers contain CD8⁺ suppressor cells that have different MHC antigens (HLA antigens in humans). However, given the enormous diversity individual MHCs, the cell bank can contain 10 or more, 50 or more, 100 or more or 1000 or more containers each containing CD8⁺ Tregs from different individuals and having different MHC antigens. CD8⁺ Tregs that partially match the patient's MHC are selected for administration to the patient.

### BRIEF DESCRIPTION OF THE DRAWINGS

FIG. 1 shows data that demonstrate that allogeneic polyclonal CD8 Tregs induced ex-vivo are more protective in vivo against a human anti-mouse graft versus host disease than CD4regs, and have a cytokine-dependent mechanism of action. Lightly irradiated immunodeficient NOD SCID IL-2R common gamma chain deficient (IL-2R γc^{-/-}) (NSG) mice were injected intravenously with 20×10⁶ human PBMC. In FIG. 1A mice were also injected IV with 5×10⁶ allogeneic CD8⁺ cells stimulated for 1 week with anti-CD3/28 beads, IL-2 (CD8_{Med}), or with TGF-β (CD8_{TGF-β}) (n>9/group). For comparison, dotted lines show NSG mice injected with 5×10⁶ CD4regs induced with anti-CD3/28 beads, IL-2, TGF-β and retinoic acid (13). In FIG. 1B mice were also injected allogeneic PBMC and 5×10⁶ autologous or allogeneic CD8_{Med} or CD8_{TGF-β}. In FIG. 1C, NSG mice were injected with similar numbers of PBMC±CD8_{Med}, or CD8_{TGF-β} in mice that received weekly injections of anti-IL-10R (0.5 mg). FIG. 1D is similar to FIG. 1B except mice received weekly injections of Alk5 TGF-βR1 signaling inhibitor (0.5 mg).

FIGS. 2A and 2B show flow cytometry data on Foxp3 expression from cells treated in accordance with the invention. In FIG. 2A naïve CD8⁺ cells were stimulated with anti-CD3/28 coated beads (1 bead per 5 cells) with IL-2 (50 U/ml)±TGF-β1 (5 ng/ml) and an alk5 TGF-βR1 signaling inhibitor (10 μM) for 5 days. The cells were permeabilized, stained for Foxp3 and analyzed by flow cytometry. In FIG. 2B. CD8 cells were stimulated as above with IL-2 50 U/ml for 4 days, washed and IL-2 added back in the amounts indicated. Foxp3 was determined after culture for 2 more days. At least 20 U/ml of IL-2 was required to sustain Foxp3 expression.

FIGS. 3A, 3B and 3C show phenotypic characteristics of CD8⁺ cells activated with anti-CD3/28 beads and IL-2. In FIGS. 3A and 3B, TGF-β has positive and negative effects on the phenotype of CD8⁺ cells stimulated with anti-CD3/28 beads and IL-2. Flow cytometry histograms comparing markers expressed by unstimulated naive CD8 cells with cells stimulated with anti-CD3/28 coated beads, IL-2 (50 U/ml)±TGF-β1, for 5 days. Isotype controls are shaded. The data for each marker shown is representative of at least three experiments. FIG. 3C demonstrates that CD8⁺ cells retain the naïve phenotype 10 days after activation. Two color scatter of CD8+ cells stained with anti-CD45RA and anti-CD45RO before, 6 days and 10 days after activation.

FIG. 4 shows the suppressive effects of CD8⁺ Tregs in vitro. FIG. 4 A demonstrates that TGF-β is not required for generation, but sustains suppressive activity. CD8 cells stimulated±TGF-β for 2 days or 5 days were mixed with allogeneic naïve CFSE-labeled CD4+CD25− cells in the ratios shown, and re-stimulated with anti-CD3/28 beads (1 bead per 2 responder cells). After 4 days dilution of CD4 CFSE was assessed by flow cytometry. The data indicates the mean±SEM of 5 separate experiments and shows equivalent suppression at 2 days, but loss of activity by CD8 cells conditioned without TGF-β. The reference control was CD4+ cells stimulated without CD8⁺ cells. FIG. 4B demonstrates that CD8⁺ Tregs preferentially target allogeneic T cells. Naïve CD8⁺ and CD4+CD25− cells isolated from peripheral blood mononuclear cells of two separate donors were stimulated with anti-CD3/28 beads, IL-2±TGF-β for 5 days and assessed for their ability to suppress the proliferation of CD4+ cells. In secondary cultures, the conditioned CD8 cells were cultured with thawed autologous or allogeneic CFSE-labeled CD4 responder cells labeled with CFSE in a 1:4 ratio and stimulated with anti-CD3/28 beads (1 bead per 2 CD4+CD25− responder cells) for 4 days. The histogram shows preferentially targeting of allogeneic CD4+ cells. FIG. 4C uses the protocol described in FIG. 4B. This experiment is representative of the variability of the suppressive effects against autologous and allogeneic CFSE-labeled CD4+ cells at various suppressor to responder ratios.

FIG. 5 demonstrates the characteristics of CD8⁺ Tregs induced with immobilized anti-CD3 and anti-CD28. FIG. 5A demonstrates that CD8⁺ Tregs are not anergic. The protocol described in FIG. 4 was used for these experiments. After generation, CD8_{Medium} and CD8_{TGF-β} were labeled with CSFE and restimulated with anti-CD3/28 beads with or without autologous or allogeneic CD4 responder cells. As shown, the CD8_{Medium} cells proliferate in response to secondary anti-CD3/28 stimulation, and this proliferation was enhanced further when CD8_{TGF-β} were cultured with responder CD4 cells. The suppressive effects of these CD8⁺ Tregs on autologous and allogeneic CD4 responder cells are shown in FIG. 4B. FIG. 5B illustrates that IL-2 does not inhibit suppressive activity. IL-2 was added in the concentrations shown to CD8⁺ Tregs mixed with CFSE-labeled responder CD4+ cells in suppressor assays in a ratio of 1:4. The experiment shown is representative of 4 similar experiments where IL-2 had no effect on CD8⁺ Treg suppressive activity. FIG. 5C shows the comparison of cytokine production between CD8_{Medium} and CD8_{TGF-β} and unstimulated CD8⁺ cells. Using protocols described above, unstimulated CD8⁺ cells and those conditioned for 5 days were cultured with phorbol myristate acetate and ionomycin for 6 hours. Brefeldin A was added for the last 5 hours. The cells were permeabilized, stained for the cytokines shown, and intracellular cytokine production assessed by flow cytometry. In each of 6 experiments performed, the conditioned CD8⁺ cells produced more IL-2 and TNF-α than unstimulated CD8⁺ cells.

FIG. 6 demonstrates that CD8⁺ Tregs lack cytotoxic activity against allogeneic T cells, unlike alloantigen-stimulated naïve CD8⁺ cells. In FIG. 6A naïve CD8 cells were cultured for 7 days with CD3/CD28 beads, IL-2 and with or without TGF-β to generate CD8 Tregs. CD8 killer cells were generated by culturing CD8 cells with allogeneic mature dendritic cells at a 30:1 T cell:DC ratio. Each CD8 cell subset was then mixed with CFSE-labeled concanavalin activated T cells from the DC donor for 4 hours, at a 30:1 effector to target cell ratio. Killing of target cells defined by CFSE-labeled cells double stained for Annexin V and 7-AAD was then determined by flow cytometry. Shown is representative example. The bar graph indicates the mean and SEM of specific killing for each CD8 subset of the four healthy donors studied. Specific cytotoxicity was determined after correction for background staining by the following formula: (observed cytotoxicity−minimum cytotoxicity)/(maximum cytotoxicity−minimum cytotoxicity)×100. P values, <0.001, compared CD8 killer with each CD8 Treg subset. The experimental results in FIG. 6 B were generated to determine whether CD8 Tregs can be converted into cytotoxic killer cells by allogeneic stimulation. CD8 Tregs were harvested at day 7, then co-cultured with irradiated allogeneic non-T cells for 6 days and examined for cytotoxity as described above. A representative example of Annexin V and 7AAD staining from one of three healthy subsets studied and a summary of the calculated CTL activity is shown in the bar graph. In these experiments the positive controls were CD8 cells stimulated with allogeneic non-T cells for 6 days.

FIG. 7 shows the flow cytometry data for baseline and maximal annexin V and 7AAD staining for the controls for the cytotoxicity assays in FIG. 6. In FIG. 7A, CD8_{Killer}, CD8_{Medium}, and CD8_{TGF-β} subsets were incubated with CFSE-labeled allogeneic concanavalin A activated T cells for 4 hours, at a 30:1 effector to target cell ratio, as described in FIG. 6. With CD8_{Medium}, and CD8 CD8_{TGF-β}, representative scatter plots show similar clusters of large target cells. However, with CD8 killer cells the cluster of large CFSE-labeled target cells was markedly decreased. FIG. 7B shows that the scatter profile of all CD8 subsets in secondary cultures was similar to primary cultures. Again, however, with CD8 killer cells the cluster of large CFSE-labeled target cells was decreased in comparison with the clusters with CD8_{Medium}, and CD8_{TGF-β}. This suggests that only the CD8 killer cells had cytolytic activity against the target cells. FIG. 7C shows the specific cytotoxicity of minimum or baseline killing and maximal killing defined as annexin V and 7-AAD double positive cells. Minimal or baseline killing was determined by annexin V and 7-AAD staining of CFSE-labeled target cells incubated alone for 4 hours, whereas maximum cytotoxicity was determined by treating target cells with permeabilization buffer (lower panel).

FIG. 8 demonstrates the role of TNFR2 and PD-L1 displayed by CD8_{Medium} and CD8_{TGF-β} in the generation and expression of suppressive activity. FIG. 8A depicts the rapid expression of both PD-L1 and TNFR2. CD8+ cells that were stimulated with anti-CD3/28 beads and IL-2±TGF-β as described above for 2 days, stained for TNFR2 and PD-L1, and sorted into TNFR2+ PD-L1+, TNFR2+ PD-L1−, TNFR2− PD-L1+, and TNFR2− PD-L1− fractions by cell sorting. Each fraction was tested for suppressive activity in vitro. An additional control was sham sorted cells. FIG. 8 B depicts the suppressive activity of each sorted CD8+ cell subset on allogeneic CD4 responder cell proliferation. The assay was set up and performed at various suppressor cell to responder cell ratios as described above. The results shown are representative of three separate experiments where the TNFR2 PD-L1 double positive cells had markedly stronger suppressive activity than sham sorted controls, and the double negative cells lacked any activity. FIG. 8C demonstrates that TNF upregulates PD-L1: CD8 cells stimulated±TGF-β as described above with TNFR-Fc (50 ug/ml) and examined for PD-L1 expression after culture for 1 or 4 days. The bars indicate the mean±SEM of three separate experiments. Blockade of TNF signaling with TNFR-Fc significantly decreased PD-L1 expression. FIG. 8D demonstrates the effects of blocking TNF signaling and anti-PD-L1 antibodies on the generation of CD8regs. CD8+ cells stimulated±TGF-β were cultured for 5 days with soluble TNFR-Fc (10-100 ug/ml) to block TNF binding to TNFR2, and with anti-PD-L1 (10-20 ug/ml). The figure shows one of three experiments with similar results. FIG. 8E demonstrates the effects of blocking TNF signaling and anti-PD-L1 antibodies on CD8 Treg suppressive activity. In these experiments similar doses of soluble TNFR2-Fc and anti-PD-L1 were added to CD8 Tregs and CD4 responder cells in the suppressor cell assay. The figure shows one of three experiments with similar results.

FIG. 9 depicts the expression levels of some of the markers which characterize CD8⁺ suppressor cells when activated under different conditions.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. Berg et al. (2002) Biochemistry, 5^{th} Ed., W. H. Freeman Pub., New York, N.Y., and Kuby Immunobiology, 6^{th} Ed., W. H. Freeman Pub., New York, N.Y. (2006), all of which are herein incorporated in their entirety by reference for all purposes. The practice of the invention also assumes an understanding of conventional immunobiological methods that are well known to the person of ordinary skill in the art of immunology. Basic information and methods can be found in Current Protocols in Immunology, editors Bierer et al., 4 volumes, John Wiley & Sons, Inc.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview

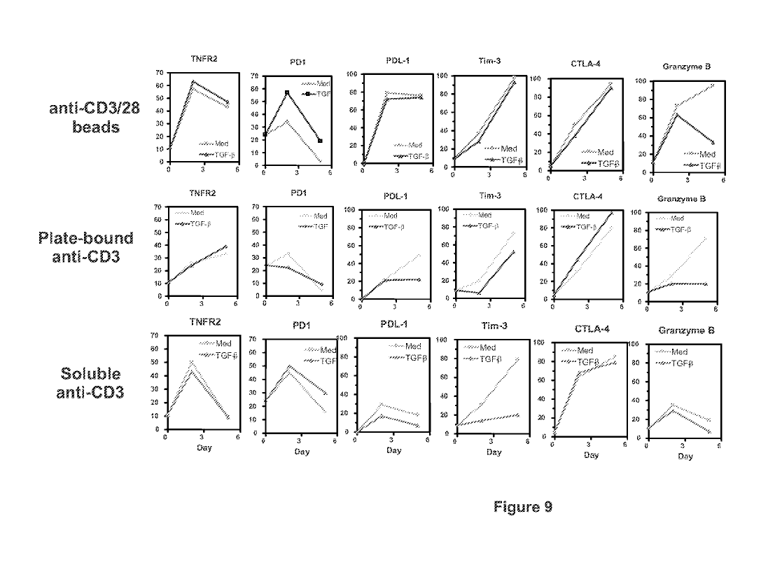

The present invention is directed to methods of generating suppressor cells by activating T cells with a suppressor-inducing composition. As used herein the term “suppressor-inducing composition” means any composition that induces naïve CD8⁺ T cells to become CD8⁺ suppressor cells. Such cells are characterized as expressing CD25, Foxp3, CTLA-4 and TNFR2. These suppressor cells also express the negative co-stimulatory markers PD-1, PD-L1 and Tim 3. See e.g. FIG. 9. CD8⁺ suppressor cells preferentially suppress allogeneic T cells as compared to autologous T cells.

In general, suppressor-inducing compositions contain one or more mitogens and one or more cytokines. Examples of mitogens include anti-CD3 antibodies, anti-CD28 antibodies and anti-CD2 antibodies as well as super-antigens such as Staphylococcal Protein A and Staphylococcal enterotoxin, the CD2 ligand, LFA-3 and Concanavalin A (Con A). Examples of cytokines include IL-2, IL-7 and IL-15. IL-7 and IL-17 are preferably used in combination with IL-2 during activation and/or expansion of the cells. A particularly preferred suppressor-inducing composition comprises anti-CD3 antibodies, anti-CD28, and/or IL-2 (or any combination thereof). The invention will be described in terms of this preferred suppressor-inducing composition. However, it is to be understood that other suppressor-inducing compositions can substitute for anti-CD3 antibodies, anti-CD28, and/or IL-2 and be within the scope of the invention.

In further aspects, methods of the invention also include applying TGF-β to CD8⁺ cells to generate suppressor cells of the invention. Suppressor cells (also known as “Regulatory cells,” “Regulatory T cells” or “Tregs”) are specialized populations of T cells that act to suppress activation of the immune system and thereby maintain immune system homeostasis and tolerance to self-antigens.

In some embodiments, the anti-CD3, anti-CD28, and IL-2 (with or without TGF-β) are applied to blood (as used herein, the term “blood” encompasses whole blood or cord blood) or a component of blood to generate suppressor cells. In certain CD8⁺ cells are first isolated from blood prior to activation to produce suppressor cells. In a preferred embodiment, suppressor cells of the invention are CD8⁺ suppressor cells.

In some aspects, suppressor cells generated in accordance with the present invention are administered to patients to treat or alleviate immune disorders. Such immune disorders include without limitation autoimmune disorders and graft versus host disease. As used herein, the term “patient” refers to any mammalian subject, including humans.

In specific aspects, the suppressor cells administered to a patient are allogeneic to that patient. As used herein allogeneic refers to the relationship between a first cell or tissue and a second cell or tissue where the first and second cells and/or tissue have different MHC antigens. For example, a CD8 suppressor cell is antigenic to a patient when the CD8⁺ Tregs have different MHC I antigens as compared to the MHC I antigens of the patient.

In certain embodiments, the suppressor cells of the invention are activated CD8⁺ cells that preferentially target allogeneic peripheral blood mononuclear cells (PBMC) to achieve suppressive activity in vivo. An advantage of suppressor cells of the present invention is that they can be administered to the patient without additional immune-suppressive therapies. Because their suppressive activities are preferentially stimulated by alloantigens, they block the patient's immune system attempt to reject them.

In further embodiments, the suppressor cells administered to a patient are generated from the cells of a donor who matches at least one set but not all of the HLA antigens in the patient.

In still further embodiments, the suppressor cells administered to a patient are contained in a blood product that is storage stable for a predetermined period of time. This blood product may be obtained from a group of blood products in which at least two of the blood products include suppressor cells that have different MHC antigens.

The present invention further encompasses blood products containing suppressor cells, including CD8⁺ suppressor cells. In specific embodiments, the blood products of the invention are stable for storage in a blood bank. In further embodiments, the blood products of the invention include CD8⁺ suppressor cells that were generated by treating blood or a component of blood with anti-CD3, anti-CD28 and IL-2. In still further embodiments, the blood is further treated with TGF-β. In certain embodiments, the blood treated in accordance with any of the above is further mixed with a blood bank storage quantity of anti-coagulant acid-citrate-dextrose solution to further stabilize the product for storage. In some embodiments, the blood products or cellular compositions can be frozen to enhance preservation and thawed prior to use.

In some aspects, the present invention provides a suppressor cell bank that comprises a collection of containers where each container contains CD8⁺ suppressor cells from a different individual. In certain embodiments, at least two of said containers contain CD8⁺ suppressor cells that have different MHC antigens (HLA antigens in humans). However, given the enormous diversity individual MHCs, the cell bank can contain 10 or more, 50 or more, 100 or more or 1000 or more containers each containing CD8 Tregs from different individuals and having different MHC antigens. CD8 Tregs that partially match the patient's MHC are selected for administration to the patient. In a preferred embodiment, the CD8⁺ suppressor cells are activated suppressor cells which have not been expanded in the presence of allogeneic MHC antigens.

### II. Methods for Generating Suppressor Cells

The present invention provides methods for generating suppressor cells. As used herein, the term “suppressor cells” is used interchangeably with “regulatory T cells” sometimes referred to as “Tregs”.

In certain aspects of the invention, naïve CD8⁺ cells are stimulated to generate suppressor cells of the invention. In some embodiments, such naïve cells are stimulated with anti-CD3, anti-CD28, IL-2, TGF-β, or any combination thereof. In other words, the naïve cells may be stimulated with any one, two, three or all of anti-CD3, anti-CD28, IL-2, TGF-β. In certain exemplary embodiments, the naïve cells are stimulated with anti-CD3 and anti-CD28 antibodies immobilized on beads, IL-2 and TGF-β to produce suppressor cells. In certain embodiments, the TGF-β is administered prior to, subsequent to or simultaneously with treatment with anti-CD3/anti-CD28 beads and IL-2. Thus as will be appreciated, any or all of the anti-CD3, anti-CD28, and IL-2 administered to produce the suppressor cells of the invention may be administered in soluble form or immobilized on a solid substrate such as a bead.

As will be appreciated, concentrations for effectively stimulating naïve CD8⁺ cells can be readily determined from the description provided herein as well as methods known in the art, including those described in U.S. Pat. Nos. 6,228,359; 6,358,506; 6,797,267; 6,803,036; 7,381,563 and 6,447,765, and U.S. application Ser. Nos. 10/772,768; 11/929,254; 11/400,950; 11/394,761; and 12/421,941, all of which are hereby incorporated in their entirety for all purposes and in particular for all teachings (including written description, figures, and working examples) directed to methods and compositions for generating suppressor cells.

In some embodiments, naïve cells are treated with anti-CD3 and/or anti-CD28 in concentration ranges of from about 0.1 to about 5.0 μg/ml. In further embodiments, concentrations of anti-CD3 and/or anti-CD28 range from about 0.2 to about 4.0, about 0.3 to about 3.0, about 0.4 to about 2.0, and about 0.5 to about 1.0 μg/ml. In addition or instead of anti-CD3 and/or anti-CD28, other T cell activators may be used to stimulate naïve cells to become suppressor cells—such T cell activators include without limitation anti-CD2, including anti-CD2 antibodies and the CD2 ligand, LFA-3, Concanavalin A (Con A), staphylococcus protein A and staphylococcus enterotoxin B (SEB).

In a related embodiment, after generation of the allogeneic suppressor cells the cells are expanded in the presence of (1) the patient's MHC antigens (HLAs in humans) and/or the MHC antigens from donor cells, tissue or organ that are to be transplanted into the patient. Peripheral blood mononuclear cells (PBMCs) can be used for this purpose. Prior to expansion, the cell population contains broadly reactive polyclonal CD8 Tregs. To survive these Tregs require continuous antigen stimulation. Therefore, exposure of the CD8 Tegs to PBMCs of the patient will select out those CD8 cells that will preferentially expand following the transplant. Such expansion concentrates the CD8⁺ Tregs that are specific for the patient's MHC I antigens. In some embodiments, the patient's PBMCs may be activated and then irradiated to enhance expansion of the CD8⁺ Tregs. In other embodiments IL-2 is also added to enhance CD8⁺ Treg expansion.

When the patient is receiving allogeneic tissue (e.g. skin), organ or bone marrow from a donor, it is preferred that the CD8⁺ Tregs be derived from the donor or a partially matched third party and expanded in the presence of MHC antigens from the patient. When CD8⁺ Tregs from the donor are used it is preferred that MHC antigens from the patient be used to expand the CD8⁺ Tregs. When partially matched CD8⁺ Tregs are used, in the case of tissue or organ transplantation, the partially matched third party CD8⁺ Tregs are preferably expanded with MHC antigens from the patient and optionally from the donor as well. In the case of bone marrow transplantation or the treatment of graft vs. host disease the partially matched third party CD8⁺ Tregs are preferably expanded with MHC antigens from the donor and optionally with MHC antigens from the patient.

By “transforming growth factor-β” or “TGF-β” herein is meant any one of the family of the TGF-βs, including the three isoforms TGF-β1, TGF-β2, and TGF-β3; see Massague, J. (1980), J. Ann. Rev. Cell Biol 6:597. Lymphocytes and monocytes produce the β1 isoform of this cytokine (Kehrl, J. H. et al. (1991), Int J Cell Cloning 9: 438-450). The TGF-β can be any form of TGF-β that is active on the mammalian cells being treated. In humans, recombinant TGF-β is currently preferred. In general, the concentration of TGF-β used in compositions of the invention can range from about 2 pg/ml of cell suspension to about 50 ng/ml. In further embodiments, the concentration of TGF-β used in compositions of the invention ranges from about 5 pg/ml to about 40 ng/ml, from about 10 pg/ml to about 30 ng/ml, from about 20 pg/ml to about 20 ng/ml, from about 30 pg/ml to about 10 ng/ml, from about 50 pg/ml to about 1 ng/ml, from about 60 pg/ml to about 500 pg/ml, from about 70 pg/ml to about 300 pg/ml, from about 80 pg/ml to about 200 pg/ml, and from about 90 pg/ml to about 100 pg/ml. In still further embodiments, the concentration of active TGF-β used in compositions of the invention are larger than the sub-picogram and picogram quantities found cultures. Such concentrations of use in the present invention include the ranges of about 1-30, 2-25, 3-20, 4-15, 5-10, 6-8 ng/ml. In still further embodiments, the concentration of TGF-β used is determined based upon endpoints such as percentage of FOXP3+ cells produced in a population of cells and stability of FOXP3 expression. Such endpoints can be determined using methods known in the art and described herein.

In some aspects, suppressor cells are generated by stimulating cells contained in blood or a component of blood. In certain embodiments, suppressor cells are generated by treating blood or a component of blood with anti-CD3, anti-CD28, IL-2, TGF-β, or any combination thereof. In further embodiments, the blood or component of blood treated in accordance with the invention contains naïve CD8+ cells.

In further embodiments, naïve CD8⁺ cells are isolated from blood or a component of blood and then treated with anti-CD3, anti-CD28, IL-2, TGF-β, or any combination thereof to produce CD8+ suppressor cells.

In some embodiments, after naïve CD8⁺ cells are treated (either while still contained blood or a component of blood or after isolation) with anti-CD3, anti-CD28, IL-2, TGF-β, or any combination thereof to produce CD8⁺ suppressor cells, the suppressor cells are further expanded to produce a larger population of suppressor cells. In certain embodiments, the suppressor cells are expanded by maintaining the cells in culture for about 1 day to about 3 months. In further embodiments, the suppressor cells are expanded in culture for about 2 days to about 2 months, for about 4 days to about 1 month, for about 5 days to about 20 days, for about 6 days to about 15 days, for about 7 days to about 10 days, and for about 8 days to about 9 days. In further embodiments, the suppressor cells are cultured in the presence of CD4+ cells. In still further embodiments, the CD4+ cells included in the culture of CD8⁺ suppressor cells are allogeneic CD4+ suppressor cells. In further embodiments, the allogeneic CD4+ suppressor cells included in the culture of the CD8⁺ suppressor cells are obtained from the patient who will eventually receive the expanded CD8⁺ suppressor cells. In other embodiments, the CD4+ suppressor cells are allogeneic to both the donor of the CD8⁺ cells and the patient who will eventually receive the expanded CD8⁺ suppressor cells.

As will be appreciated, cultures of suppressor cells may further include cell culture media and any additives known in the art for maintenance of the cell culture. Cultures of suppressor cells may also include IL-7, IL-15, a retinoic acid derivative such as all trans retinoic acid or a ppAR agonist (including without limitation ciglitizone) or any combination thereof.

Suppressor cells of the invention preferentially target allogeneic T cells over autologous cells when administered to a patient. As will be discussed in further detail herein, this characteristic of suppressor cells of the invention make them particularly well suited for the treatment of immune disorders.

In further aspects, the suppressor cells of the invention are able to maintain suppressive activity in the presence of IL-2.

In some aspects, the suppressor cells of the present invention express one or both of TNFR2 and PD-L1.

In further aspects, the methods of the present invention results in a population of suppressor cells that comprises at least 50% CD8+ suppressor cells. In further embodiments, the population of suppressor cells comprises about 50-100, 55-95, 60-90, 65-85, 70-80% CD8⁺ suppressor cells. In still further embodiments, the population of suppressor cells comprises at least 50%, 55%, 60%, 65%, 70%, 75%, 80%, 85%, 90%, 91%, 92%, 93%, 94%, 95%, 96%, 97%, 98%, 99%, 100% CD8⁺ suppressor cells.

### III. Methods for Using Suppressor Cells of the Invention

In a preferred aspect, the present invention provides methods for administering suppressor cells generated as described herein to patients to prevent and/or treat an immune disorder. “Immune disorder” as used herein encompasses autoimmune diseases as well as aberrant or undesired immune responses (such as graft rejection and graft versus host disease). Immune disorder also encompasses chronic immune disease triggered by a virus or other infectious agent or toxin.

In certain aspects, the present invention provides methods for administering CD8⁺ suppressor cells to a patient to treat or alleviate an immune disorder. In further aspects, the CD8⁺ suppressor cells are allogeneic to the patient.

In further aspects, the present invention provides methods for resetting the patient's immune system by administering allogeneic CD8⁺ suppressor cells. Normally, the antigen presenting cells of the immune system are tolerogenic, and the T and B cells are non-responsive to self antigens. In autoimmune diseases, the antigen-presenting cells become immunogenic causing T and B cells to become responsive to self antigens. By resetting the immune system, the antigen-presenting cells convert from immunogenic to tolerogenic causing the responsive immune T cells and B cells again become non-responsive. Conventional therapies for resetting the immune system to restore the tolerogenic circuit involve autologous stem cell transplantation. This approach requires massive depletion of T and B cells followed by reconstitution with autologous stem cells. Administration of suppressor cells in accordance of the present invention has the advantage of providing the beneficial effects of resetting the immune system without the risk of morbidity associated with autologous stem cell transplantation.

In further aspects, the present invention provides methods for resetting the patient's immune system by administering allogeneic CD8⁺ suppressor cells. Normally, the antigen presenting cells of the immune system are tolerogenic, and the T and B cells are non-responsive to self antigens. In autoimmune diseases, immune cells become responsive to self antigens. By resetting the immune system, the antigen-presenting cells become immunogenic causing T cells and B cells to become responsive again become non-responsive. Conventional therapies for resetting the immune system to restore the tolerogenic circuit involve autologous stem cell transplantation. This approach requires massive depletion of T and B cells followed by reconstitution with autologous stem cells. Administration of suppressor cells in accordance of the present invention has the advantage of providing the beneficial effects of resetting the immune system without the risk of morbidity associated with autologous stem cell transplantation.

In preferred embodiments, suppressor cells used to treat or prevent immune disorders are generated using any of the methods described herein. In certain embodiments, the suppressor cells are generated using a method in which blood or a component of blood that contains naïve CD8⁺ cells is contacted with anti CD-28, anti-CD3 and IL-2 to activate the naïve CD8⁺ cells to become suppressor cells. In specific embodiments, the anti-CD3 and anti-CD28 is applied using anti-CD3/28 beads. In further embodiments, the naive CD8⁺ cells are further treated with TGF-β prior to, simultaneously with, or subsequent to treatment with anti CD-28, anti-CD3 and IL-2.

In further embodiments, the suppressor cells are generated from blood (or a component of blood) or naïve CD8⁺ cells isolated from a blood sample obtained from a donor. The donor may match the patient for one set of HLA antigens or more than one set of HLA antigens up to but not including all HLA antigens. An advantage of the methods and compositions of the present invention is that suppressor cells generated from a donor who is less than a perfect HLA antigen match to the patient can still be used to treat the patient without triggering a rejection of the suppressor cells by the patient's own immune system.

In still further embodiments, the suppressor cells preferentially inhibit allogeneic T cells in the patient over autologous cells. This is of particular use in the treatment of immune disorders, because the suppressor cells of the invention can be administered to patients without co-administration of other immunosuppressive therapies.

In yet further embodiments, the allogeneic suppressor cells that are administered to a patient to prevent or treat immune disorders are contained in a blood product. As is discussed in further detail herein, blood products of the invention may be storage stable and/or obtained from a suppressor cell bank.

In further aspects and in accordance with any of the above, patients receiving CD8+ suppressor cells of the invention are further administered low-dose IL-2 to maintain suppressor cell activity. This low-dose IL-2 may be administered prior to, simultaneously with or subsequent to administration of the allogeneic CD8⁺ suppressor cells. In further embodiments, the low-dose IL-2 may be administered once or multiple times over a period of hours, days or weeks to the patient receiving CD8⁺ suppressor cells. In still further embodiments, the low-dose IL-2 is in a concentration of about 1 million IU, 1.5 million IU, or 3 million IU per administration. In yet further embodiments, the low-dose IL-2 is in a concentration of about 0.5-4, 1-3.5, 1.5-3.0, 2.0-2.5 million IU per administration. In further embodiments IL-7 may be used instead of IL-2.

### IV. Blood Products and Compositions Comprising Suppressor Cells

In some aspects, the present invention encompasses blood products and compositions that contain suppressor cells. In further aspects, the blood products and compositions of the invention comprise CD8⁺ suppressor cells.

A preferred composition comprises suppressor cells generated by activating naïve CD8⁺ cells by treating blood, a component of blood comprising naïve CD8+ cells or isolated naïve CD8⁺ cells with anti-CD3, anti-CD28 and IL-2 and expanding the activated CD8⁺ cells by culturing in the presence of at least first and second different MHC antigens which are allogeneic to the MHC antigens of said CD8+ cells.

As discussed herein, suppressor cells of the present invention can be administered to a patient to prevent or treat an immune disorder. One advantage of the suppressor cells of the present invention is that these cells can be allogeneic to the patient. In certain instances, these allogeneic suppressor cells are matched to the patient in at least one set of HLA antigens. These allogeneic suppressor cells can be stored as a collection of suppressor cells, for example in blood products that are storage stable. In some embodiments, blood products of the invention are stored in a blood product bank. Blood product banks of the invention comprise a cache or bank of blood products that contain suppressor cells.

In some embodiments, blood products of the invention include blood or components of blood that have been treated with anti-CD3, anti-CD28, IL-2, TGF-β, or any combination thereof to produce suppressor cells contained within the blood or component of blood.

In further aspects, the blood products of the invention are storage stable for a predetermined period of time. In exemplary embodiments, the blood products of the invention are storage stable for at least 1 day, 1 week, 1 month, 1 year, 2 years, 5 years. In further embodiments, the blood products of the invention are stored for about 1-60, 2-55, 3-50, 4-45, 5-40, 6-35, 7-30, 8-25, 9-20, 10-15 days. Such storage can be at temperatures suitable for routine blood storage, including without limitation storage at 1-6° C. in a unit or container appropriate for maintaining temperature stability and sterility.

Blood products of the invention can be stored within a group of blood products, where at least two of the blood products in the group include suppressor cells that have different HLA antigens. These blood products may further include a blood bank storage quantity of additives known in the art to be useful in the storage of blood and blood products for later administration to patients. Such additives may include without limitation acid-citrate-dextrose, citrate-phosphate-dextrose, citrate-phosphate-double-dextrose, and citrate-phosphate-dextrose-adenine. These additives may be included as a solution to the blood products.

In further embodiments, CD8⁺ suppressor cells in blood products of the invention inhibit allogeneic cells when that blood product is administered to a patient.

In some aspects, the present invention provides a suppressor cell bank that includes a collection of containers, and each container contains CD8⁺ suppressor cells. In certain embodiments, at least two of those containers contain CD8⁺ suppressor cells that have different MHC antigens from each other. In further embodiments, single containers may also themselves contain mixtures of suppressor cells comprising different sets of MHC antigens. Such cell banks can be stored at blood bank temperatures or frozen to enhance preservation.

In some aspects, the blood products of the invention include naïve CD8+ cells. As with the blood products containing suppressor cells, the blood products containing naïve cells are storage stable. In further embodiments, the naïve CD8+ cells are stimulated to become suppressor cells using any of the methods and compositions discussed herein just prior to administration of the blood product to the patient.

In further aspects and in accordance with any of the above, the blood products of the invention are sterilized prior to storage and/or prior to administration to a patient. In further embodiments, the blood products are tested subsequent to sterilization to assess whether suppressive activity is retained through the sterilization process. Such assessment can be made using assays known in the art and described herein.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Materials and Methods

### Mice

NOD/scid/IL2r common γ chain^{-/- (NSG) mice were obtained from Jackson Laboratory (Bar Harbor, Me.). The mice were bred and housed under specific pathogen-free conditions in microisolator cages and given unrestricted access to autoclaved food and sterile water. Animals of both sexes were used for experiments at} 8-12 weeks of age. The mice received a single dose of 150 cGy gamma irradiation from a linear accelerator before injection of human PBMC on the same day. All experiments were performed according to the guidelines of the Institutional Animal Committee of the University of Southern California.

### Monoclonal Antibodies, Cytokines and Cytokine Antagonists Used

The following FITC, PE, Cyc or APC conjugated human antibodies were used for flow cytometric analysis: From BD Pharmingen (San Diego, Calif.): CD3(HIT3a), CD4 (RM4-5), CD28 (CD28.2), CD45RA (L48), CD45RO (UCHL1), CD122 (Mik-β3), CD86 (2331[FUN1]), CD103 (Ber-ACT8), CD274, PD-L1 (M1H1), CTLA-4 (BNI3), HLA-DR (G46-6), Granzyme A (CB9), Granzyme B (GB11), mouse IgG1 (MOPC-21), IgG2a (G155-178), IgG2b (27-35), from Biolegend, (San Diego, Calif.): CD8 (SK1), CD25 (M-A251), PD-1 (EH12.2H7), CD274, PD-L1 (29E.2A3), Tim3 (F38-2E2), from eBiosciences (San Diego, Calif.): Foxp3 (206D), and from R&D Systems, Inc (Minneapolis, Minn.): TNF-RII (22235). We obtained unconjugated PD-1 (MIH4) and PD-L1 (MIH1) and CTLA-4 (BNI3.1) from BD Pharmingen as a generous gift from Noel Warner. TNFR-Fc (Enbrel) was obtained from Amgen, (Thousand Oaks, Calif.). Other agents purchased from BD Pharmingen included recombinant human IL-2 (MQ1-17H12), IFN-γ (B27), from HumanZyme (Chicago, Ill.); recombinant human TGF-β1, from Invitrogen (Carlsbad, Calif.): anti-human CD3/CD28-conjugated Dynabeads, carboxyfluorescein succinimidyl ester (CFSE), and AIM-V serum-free medium from GIBCO Invitrogen, Life Technologies, (Grand Island, N.Y.); RPMI 1640 medium, Cellgro Mediatech, (Manassas, Va.) Fetal Bovine Serum (FBS) Atlanta Biologicals, (Lawrenceville, Ga.).

Isolation of Human nTregs and Generation of Human iTreg Cells Ex Vivo

PBMC were prepared from heparinized venous blood of healthy adult volunteers by Ficoll-Hypaque density gradient centrifugation. All protocols that involved human blood donors were approved by the IRB at the University of Southern California. T cells were prepared by E rosetting and negative selection of non-T cells as described previously to a purity of >95% (S. Yamagiwa et al., A role for TGF-beta in the generation and expansion of CD4+CD25+ regulatory T cells from human peripheral blood. Journal of immunology 166, 7282 (Jun. 15, 2001)). The T cells were incubated with unconjugated mouse anti-CD4 (OKT4), anti-CD45RO (UCHL1), anti-HLA-DR (L243), and anti-CD11b (OKM1) (American Type Tissue Culture Collection, Bethesda, Md.) and depleted with goat anti-mouse IgG coated beads (Dynabeads, Life Technologies, Grand Island, N.Y.). This isolation procedure was repeated to increase the purity to >90%. The naïve CD8 cells were stimulated with CD3/28 beads at 1:5 ratio (one beads in 5 cells)+rhlL-2 (50 U/ml) CD8_{Medium} with TGFβ1 (5 ng/ml) CD8_{TGFβ} in AIM-V serum-free medium containing Hepes buffer (10 mM), sodium pyruvate (1 mM), glutamine and penicillin and streptomycin in 24 or 48 well plates. On day 3, cells were split and 30-50 U/ml IL-2 and fresh culture medium was added to the wells. The cells were harvested for on days 5 or 6. IL-2 50 U/ml IL-2 was added one day before harvest and the beads were removed. In experiments to assess cytokine production, the CD8 cells were stimulated with PMA and ionomycin for 6 hours. Brefeldin A was added one hour later and the cells were permeabilized (Fix and Perm Kit™ (BD) and stained for IL-2, IFN-γ, TNF-α and IL-17. Intracellular cytokine production was determined by flow cytometry. In some experiments we determined the effect of anti-PD-L1 and TNFR-Fc, in the doses indicated, on the generation of CD8regs and their suppressive activity.

### Flow Cytometry

The effect of the CD8+ cell conditioning procedures on their phenotype was assessed by comparative studies with fresh, unstimulated cells. Each subset was stained with mAbs to markers indicated above, and analyzed on a FACS Calibur flow cytometer using Cell Quest Software (Becton Dickinson). For Foxp3, CTLA-4 and granzymes A and B the cells were also permeabilized for intracellular staining. Histograms also showing isotype control staining were prepared using FloJo Software (Treestar Inc. Ashland, Oreg.).

Suppressive assays of CD4⁺ Treg cells in vitro and in vivo CD8_{Medium} or CD8_{TGFβ} (Tregs) were added to 1.5×10⁵ autologousor allogeneic CD4+ CD25 depleted cells (responder T cells) in ratios of 1:2, 1:4 and 1:8 in 96 well flat bottomed plates (Greiner Bio-one (Monroe, N.C.)). The cells were stimulated with anti-CD3/28 beads (bead:responder ratio 1:2 and 1:4) for 3 to 4 days in RPMI 1640 culture medium (Cellgro Mediatech) with 10% fetal bovine serum (Atlanta Biologicals, Lawrenceville, Ga.) The CD4 responder cells, and in some experiments the CD8 cells were labeled with CFSE as previously described (D. A. Horwitz et al., Natural and TGF-beta-induced Foxp3(+)CD4(+) CD25(+) regulatory T cells are not mirror images of each other. Trends in immunology 29, 429 (September, 2008)). Antigen-presenting cells were omitted. Cell division was monitored by CFSE dilution. Suppressive activity was the calculated as the percentage of cycling CD4 responder cells cultured with CD8 cells divided by the percentage of cycling responder cells cultured alone×100.

The model to assess suppressor activity in vivo was the protection of immunodeficient NSG mice from a rapidly fatal human anti-mouse GVHD as described previously (T. Mutis et al., Human regulatory T cells control xenogeneic graft-versus-host disease induced by autologous T cells in RAG2−/−gammac−/− immunodeficient mice. Clin Cancer Res 12, 5520 (Sep. 15, 2006)). Twenty ×10⁶ human PBMC with 5×10⁶ allogeneic or autologous CD8_{Medium} or CD8_{TGFβ} in 0.2 ml were injected IV into the tail vein of NSG mice sublethally irradiated with 150 cGy. The positive control was mice injected with PBMC only, and the negative control was mice injected with PBMC and un-stimulated CD8 cells. In some experiments anti-PD-1, anti-PD-L1 or anti-CTLA-4 (0.5 mg) were incubated with the CD8reg/PBMC mixture for 2 hours before injection as described by others (S. Amarnath et al., Regulatory T cells and human myeloid dendritic cells promote tolerance via programmed death ligand-1. PLoS Biol 8, e1000302 (February, 2010)). The animals were weighed every 2 to 3 days and euthanized when they lost 20% of their original weight. In other experiments the effect of decreasing IL-10 and TGF-β signaling on the protective effects of CD8regs was determined by injecting the mice IP with the ALK5 TGF-βR1 inhibitor (LY-364947, Sigma-Aldrich, St. Louis, Mo.) and anti IL-10R (Taconic, Germantown, N.Y., clone:YL03.1B1.39-34ABS), 0.5 mg IP weekly.

### Cytotoxicity Assay.

Cytotoxic killer cells were generated by stimulating naïve CD8 cells with allogeneic monocyte-derived mature DCs (D. W. O'Neill, N. Bhardwaj, Differentiation of peripheral blood monocytes into dendritic cells. Current protocols in immunology/edited by John E. Coligan . . . [et al.] Chapter 22, Unit 22F 4 (July, 2005)) at a 30:1 ratio (T cells: DCs). Cells were harvested at day 6 or 7 of culture, and spun through a density gradient to remove dead cells. Target cells were total T cells from the allogeneic donor activated with concanavalin A (Sigma) 5 μg/ml for 4 days. We used three color flow cytometry based upon a method previously described to determine cytotoxic activity (E. Derby et al., Three-color flow cytometric assay for the study of the mechanisms of cell-mediated cytotoxicity. Immunology letters 78, 35 (Aug. 1, 2001)). Each CD8 subset was incubated with CFSE-labeled allogeneic concanavalin A blasts for 4 hours, at a 30:1 effector to target cell ratio. Cytotoxicity was determined by staining of Annexin V and 7-AAD using a kit supplied by eBioscience and following the manufacturers instructions. Target cells killed were double stained by Annexin V and 7-AAD, and specific cytotoxicity was determined after correction for background staining by the following formula: (observed cytotoxicity−minimum cytotoxicity)/(maximum cytotoxicity−minimum cytotoxicity)×100.

### Statistical Analysis:

Flow cytometry and cytokine data were analyzed using Student 2-tailed t-tests using Graph Pad Prism Software. Comparison values of p<0.05 were considered statistically significant. Survival was determined using the Kaplan-Meier test.

### Example 1

### CD8+ Cells Stimulated with Anti-CD3 and Anti-CD28 Coated Beads have Strong Protective Activity in Humanized Mice and Preferentially Target Allogeneic T Cells

Because in vitro suppressor assays may not reflect the protective effects of Tregs in vivo, we elected to use immunodeficient mice to study the suppressive effects of human naïve CD8+ cells stimulated with anti-CD3/28 coated beads, IL-2±TGF-β. Since first reported by Mutis and co-workers (T. Mutis et al., Human regulatory T cells control xenogeneic graft-versus-host disease induced by autologous T cells in RAG2−/−gammac−/− immunodeficient mice. Clin Cancer Res 12, 5520 (Sep. 15, 2006)) we and others have used this assay to investigate the protective effects of expanded endogenous CD4+CD25+ Foxp3+ Tregs and CD4 iTregs induced ex-vivo with IL-2, TGF-β and retinoic acid (L. Lu, et al., Characterization of protective human CD4CD25 FOXP3 regulatory T cells generated with IL-2, TGF-beta and retinoic acid. PloS one 5, e15150 (2010)). Since these mice cannot reject human T cells, they develop an ultimately fatal graft-versus host disease. We and others have reported that endogenous and ex-vivo generated CD4regs enhance survival by 50 to 100%. See Table 1.

TABLE 1
Comparison of human CD4 expanded endogenous Tregs and induced Tregs with CD8
Tregs in preventing human anti-mouse GVHD in immunodeficient mice

| | | 50% survival | | |
|---|---|---|---|---|
| | | (Days) | | |
| | Treg/PBMC | PBMC | | Percent increase |
| Treg subset | ratio | PBMC + Tregs | | in survival |
| June C (2008)¹ | | | | 100+ |
| Expanded CD4 nTregs (artificial APC | 1:5 | 25 | 50+ | |
| [aAPC}, IL-2 and rapamycin) | | | | |
| Fowler D (2010)² | | | | 102 |
| Expanded CD4 nTregs (IL-2, TGF-β | 1:20 | 37 | 75 | |
| Rapamycin) | | | | |
| Blazar B (2011)³ | | | | |
| Expanded CD4 nTregs (aAPC, IL-2) | | | | |
| (x1) | 1:1 | 39 | 55 | 41 |
| (x3) | 1:2 | 46 | 53 | 15 |
| (x4) | 1:2 | 46 | 60 | 30 |
| Blazar B (2011)⁴ | | | | |
| CD4 iTregs (IL-2, TGF-β, Rapa) | 1:3 | 25 | 45 | 80 |
| CD4 nTregs (x1) | 1:3 | 38 | 55 | 45 |
| CD4 nTregs (expanded 40 days) | 1:1 | 48 | 62 | 29 |
| Horwitz DA (2010)⁵ | | | | |
| CD4 iTregs (IL-2, TGF-β, atRA) | 1:4 | 11 | 24 | 118 |
| Expanded CD4 nTregs (x1) | 1:4 | 11 | 20 | 80 |
| Horwitz DA (Present Report) | | | | |
| CD8 iTregs (IL-2) | 1:4 | 11 | 44 | 300 |
| CD8 iTregs (IL-2, TGF-β) | 1:4 | 11 | 42 | 282 |

¹R. G. Carroll et al., Distinct effects of IL-18 on the engraftment and function of human effector CD8 T cells and regulatory T cells. PloS one 3, e3289 (2008).
²S. Amarnath et al., Regulatory T cells and human myeloid dendritic cells promote tolerance via programmed death ligand-1. PLoS Biol 8, e1000302 (Febraury 2010).
³K. L. Hippen et al., Massive ex vivo expansion of human natural regulatory T cells (T(regs)) with minimal loss of in vivo functional activity. Sci Transl Med 3, 83ra41 (May 18, 2011).
⁴K. L. Hippen et al., Generation and large-scale expansion of human inducible regulatory T cells that suppress graft-versus-host disease. Am J Transplant 11, 1148 (June 2011).
⁵L. Lu, et al., Characterization of protective human CD4CD25 FOXP3 regulatory T cells generated with IL-2, TGF-beta and retinoic acid. PloS one 5, e15150 (2010).
Abbreviations: aAPC, artificial antigen-presenting cells; atRA, all trans retinoic acid; nTregs, endogenous Tregs (both thymus-derived natural and those induced in vivo); iTregs, Tregs induced ex-vivo

Unless aAPC are indicated, CD4+ cells were stimulated with anti-CD3/28 coated beads.

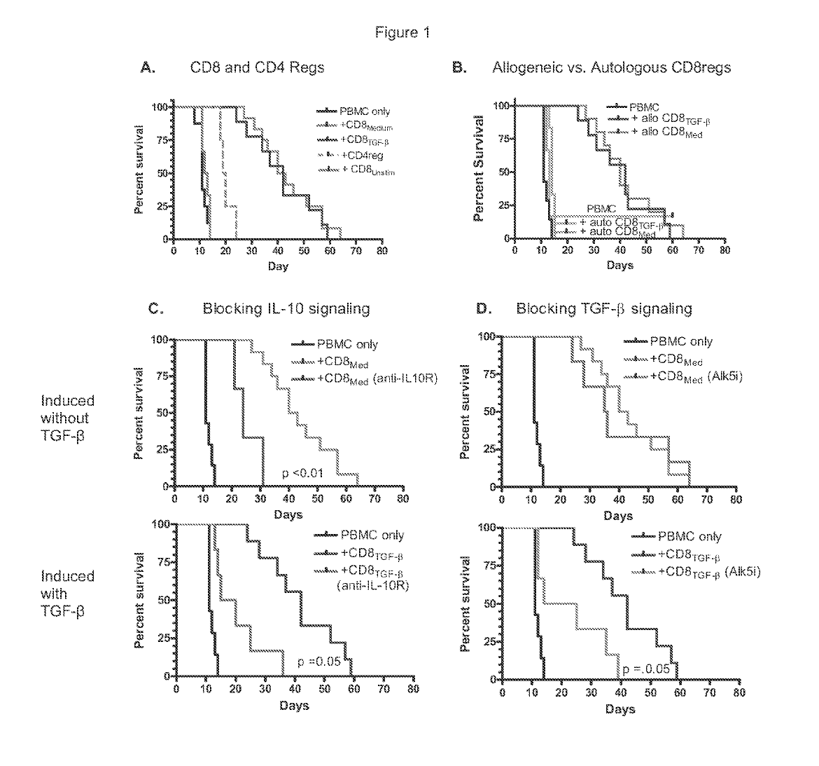

We previously reported that intravenous transfer of twenty million human PBMC into lightly irradiated NOD SCID IL-2R common gamma chain deficient (IL-2R γc^{-/-}) (NSG) mice resulted in rapid engraftment of human T cells in the lungs, liver, bone marrow and spleen leading to death of the animal in two weeks (L. Lu, et al., Characterization of protective human CD4CD25 FOXP3 regulatory T cells generated with IL-2, TGF-beta and retinoic acid. PloS one 5, e15150 (2010)). This model was also used in the present study. Where we had reported that 5 million CD4iTregs doubled survival (dotted line), transfer of a similar number of conditioned CD8+ cells quadrupled the survival of the mice (FIG. 1A). Unlike CD4regs which are TGF-β dependent, conditioning CD8+ cells without this cytokine had similar protective effects. Control untreated CD8+ cells completely lacked protective activity. Adding retinoic acid to IL-2 and TGF-β did not enhance survival further (result not shown).

Four mice were sacrificed at days 59 and 60 for histologic inspection. The characteristic liver inflammatory lesions were not observed, but there were some mononuclear infiltrates in the lungs. Some human CD8+ cells were observed in the spleen and bone marrow, but not CD4+ cells. Thus, the CD8+ cells had blocked the marked engraftment of human T cells in these mice and greatly prolonged the life of these animals. As will be shown below, this protective activity could not be explained by cytolytic effects. These CD8+ cells can be, therefore, called suppressor/regulatory cells or CD8regs.

We then compared the effects of CD8regs on autologous or allogeneic PBMC transferred to the mice. Because of the large numbers of human PBMC needed to cause rapid GVHD, we used PBMC allogeneic to the CD8+ cells in the initial experiments. Previously, in our studies with CD4regs, we had observed the protective effects were similar on both (L. Lu, et al., Characterization of protective human CD4CD25 FOXP3 regulatory T cells generated with IL-2, TGF-beta and retinoic acid. PloS one 5, e15150 (2010)). However, this was not the case with CD8regs we had generated. With one exception, neither CD8regs generated with TGF-β nor without this cytokine could increase the survival of mice that had received autologous T cells (See FIG. 1B). Thus, the CD8regs appeared to depend on alloantigen stimulation provided by their target cells for their protective effects.

Since we had observed that blocking IL-10 and TGF-β signaling abrogated the protective effects of iCD4regs in a mouse chronic GVHD (A. Horwitz et al., Natural and TGF-beta-induced Foxp3(+)CD4(+) CD25(+) regulatory T cells are not mirror images of each other. Trends in immunology 29, 429 (September, 2008)), similar studies were conducted with this model. We found that weekly injections of anti-IL-10R antibodies inhibited the protective effects of CD8regs conditioned with and without TGF-β (See FIG. 10). One difference between the two CD8reg subsets was that blocking TGF-β signaling through TGF-βR1 partially inhibited the protective effects of CD8regs conditioned with TGF-β, but had no effect on CD8regs conditioned without this cytokine (See FIG. 1D).

### Example 2

### CD8+ Cells Stimulated with Anti-CD3/28 Beads Strongly Express IL-2Ral3 Chains, TNFR2, Negative Co-Stimulatory Molecules Including PD-L1, and TGF-β Dependent Foxp3

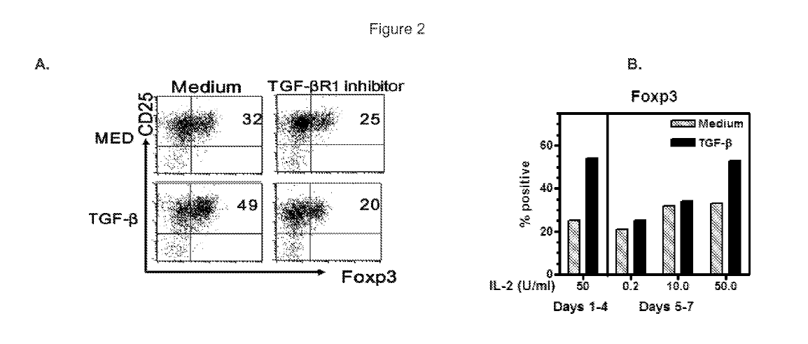

Like CD4+CD25+ Foxp3+ Tregs (L. Lu, et al., Characterization of protective human CD4CD25 FOXP3 regulatory T cells generated with IL-2, TGF-beta and retinoic acid. PloS one 5, e15150 (2010)), CD8+ cells stimulated with anti-CD3/28 beads strongly express CD25 and CD122. Thirty to 40% of stimulated CD8+ cells displayed Foxp3. This was enhanced by adding TGF-β and adding an alk5 TGF-βR1 signaling inhibitor decreased Foxp3 to baseline levels expressed by activated CD8 cells (˜20%) (See FIG. 2A). TGF-β enhanced Foxp3, however, was not stable. Sustained high levels required the addition of >20 U/ml IL-2 every three days. As shown in FIG. 2B, Foxp3 expression decreased if lower amounts were added. Thus, as demonstrated for CD4regs, both IL-2 and TGF-β have important roles in Foxp3 expression by CD8+ cells (M. O. Li et al., Transforming growth factor-beta controls development, homeostasis, and tolerance of T cells by regulatory T cell-dependent and -independent mechanisms. Immunity 25, 455 (September, 2006)).

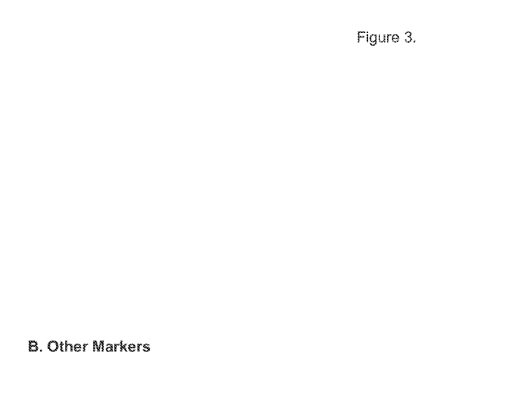

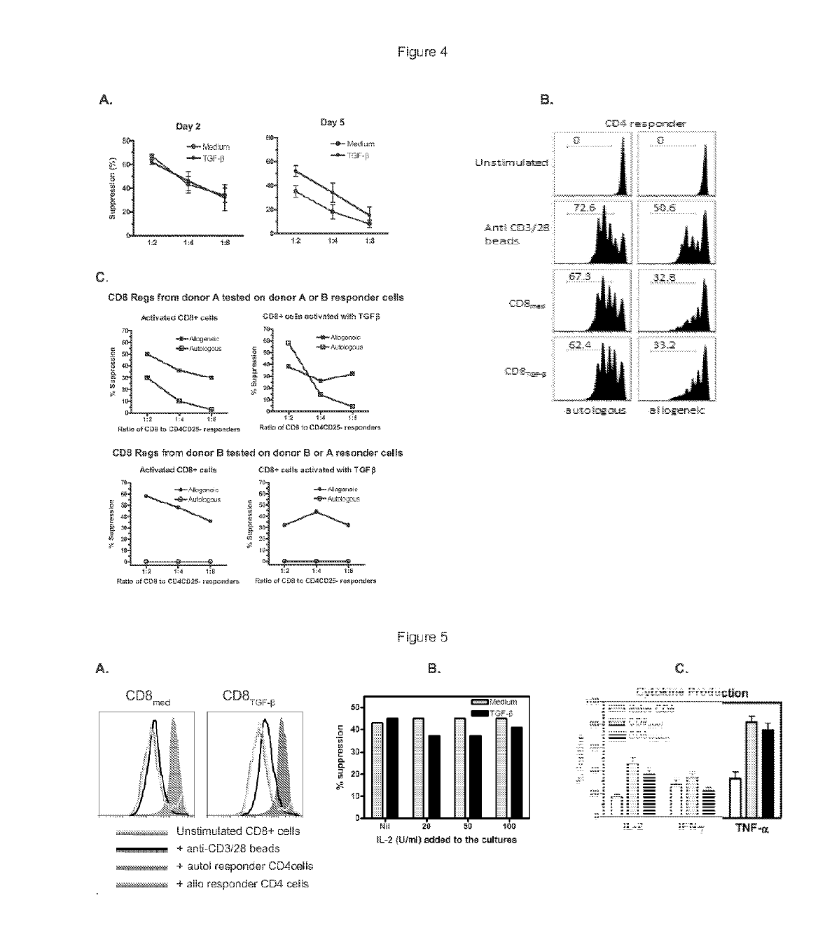

Anti-CD3/28 CD8+ stimulated cells also express TNFR2, and the negative co-stimulatory molecules CTLA-4, PD-1, PD-L1, Tim-3 (See FIG. 3A). Although >90% of the activated CD8+ cells rapidly expressed PD-L1 with anti-PD-L1 mAb clone 29E.2A3, the maximum values were somewhat less with clone M1N1. The addition of TGF-β enhanced only PD-1. As reported by others TGF-β, strongly upregulates CD103 on CD8 cells (D. Wang et al., Regulation of CD103 expression by CD8+ T cells responding to renal allografts. Journal of immunology 172, 214 (Jan. 1, 2004)). In addition, TGF-β attenuated positive co-stimulatory molecules induced by anti-CD3 that included HLA-DR, CD80 and CD86 (FIG. 3B). Moreover, although anti-CD3 stimulated CD8+ cells can become cytolytic (R. De Jong et al., Generation of alloreactive cytolytic T lymphocytes by immobilized anti-CD3 monoclonal antibodies. Analysis of requirements for human cytolytic T-lymphocyte differentiation. Immunology 70, 357 (July, 1990)), TGF-β down-regulated granzyme expression as shown here (See FIG. 3B). While CD8regs previously described are generally experienced CD45RO+ memory cells, few of our CD45RA+ starting population have become CD45RO+ during the conditioning process, especially with TGF-β (See FIG. 3B). Therefore, unlike CD4regs now undergoing clinical trials which have been extensively expanded ex-vivo indicated in Table 1, these CD8regs should have strong proliferative potential after transfer.

### Example 3

### CD8 Tregs Sustained by TGF-β Preferentially Target Alloqeneic T Cells

Similar to the in vivo studies, TGF-β was not needed for the inhibitory effects of anti-CD3/28 activated CD8 cells. FIG. 4A shows that within 2 days after activation, CD8+ cells had developed strong in vitro suppressive activity. However, by day 5 the suppressive activity by CD8+ cells stimulated without TGF-β began to decline while those with added TGF-6 did not. A likely explanation for this effect is the ability of TGF-β to protect CD8 cells from apoptosis (M. O. Li et al., Transforming growth factor-beta controls development, homeostasis, and tolerance of T cells by regulatory T cell-dependent and -independent mechanisms. Immunity 25, 455 (September, 2006)).

Also consistent with the in vivo protective effects described above was that suppressive activity in vitro against allogeneic CD4+ cells was greater than against autologous cells (See FIGS. 4B and 4C. This characteristic distinguishes these CD8regs from other polyclonal Tregs. Their activity against autologous CD4+ cells was donor variable. With some CD8regs had suppressive effects at high Treg to Tresponder ratios, but with other donors they completely lacked activity against autologous CD4+ cells as shown in FIG. 4C.

### Example 4

### CD8 Tregs are not Anergic and Lack Cytolytic Activity

One of the characteristic features of Foxp3+CD4regs is that they are anergic and one of their suppressive mechanisms is consuming IL-2 produced by other T cells (P. Pandiyan et al., CD4+CD25+ Foxp3+ regulatory T cells induce cytokine deprivation-mediated apoptosis of effector CD4+ T cells. Nature immunology 8, 1353 (December, 2007)). While the addition of IL-2 abolishes the suppressive activity of CD4regs (E. M. Shevach, Biological functions of regulatory T cells. Adv Immunol 112, 137 (2011)), IL-2 had no effect on the suppressive activity of CD8 cells (FIG. 5B). Moreover, unlike CD4regs which cannot produce IL-2 or other cytokines (E. M. Shevach, Biological functions of regulatory T cells. Adv Immunol 112, 137 (2011)), CD8regs retained their ability to produce IL-2, IFN-γ and TNF-α. In fact, the percentage of IL-2 and TNF-α producing cells increased following conditioning (FIG. 5C). The ability to produce IL-2 and proliferate while inhibiting other T cells contrasts the suppressive properties of these CD8regs from CD4regs.

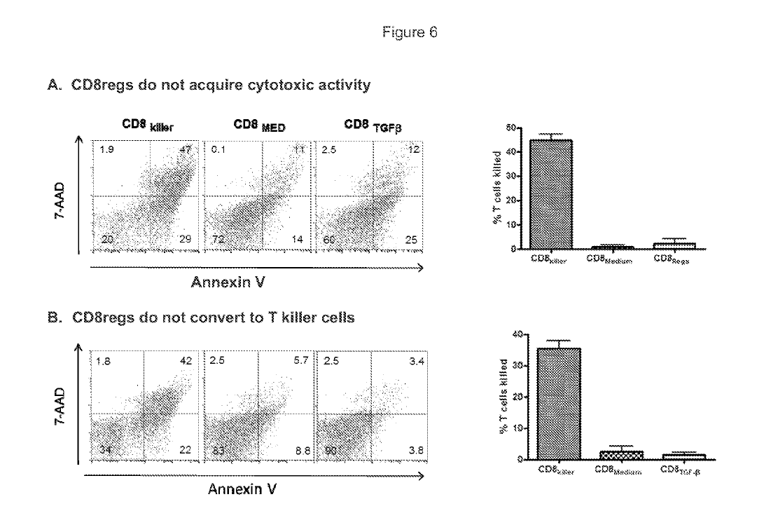

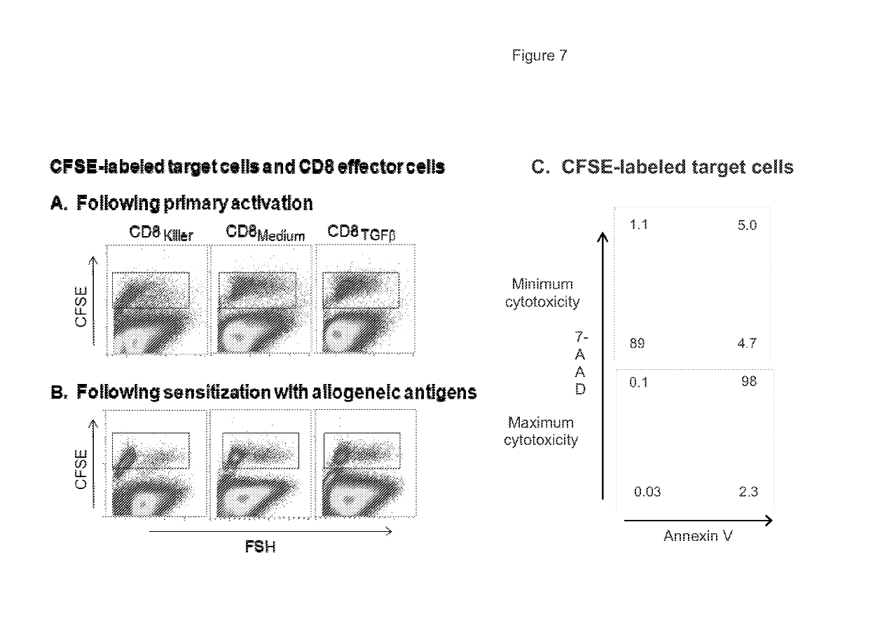

One of the principal activities of CD8+ cells is to recognize and kill MHC non-identical cells. It was especially important, therefore, to investigate the possible cytotoxic activity of these CD8regs. We used a method similar to that described in our report that human naïve CD4+ cells alloactivated with IL-2 and TGF-β developed the capacity to suppress CD8+ cells from becoming killer cells (S. Yamagiwa et al., A role for TGF-beta in the generation and expansion of CD4+CD25+ regulatory T cells from human peripheral blood. Journal of immunology 166, 7282 (Jun. 15, 2001)). We stimulated CD8+ cells with allogeneic non-T cells or mature dendritic cells, and assessed killer activity against CFSE-labeled allogeneic concanavalin A activated T cells. Instead of using radioisotopes, however, we documented apoptotic cell death by target cells stained by annexin V and 7AAD (E. Derby et al., Three-color flow cytometric assay for the study of the mechanisms of cell-mediated cytotoxicity. Immunology letters 78, 35 (Aug. 1, 2001)). The FACS profile and baseline staining is shown in Supplementary FIG. 1. Target cell death following a 4 hour incubation with CD8 subsets, and specific cytotoxic activity calculated by a formula indicated in the Methods, is shown in FIG. 6. While we expected that CD8+ cells stimulated with TGF-β would not develop killer activity (M. O. Li at al., Transforming growth factor-beta controls development, homeostasis, and tolerance of T cells by regulatory T cell-dependent and -independent mechanisms. Immunity 25, 455 (September, 2006)), we were surprised to find that neither CD8+ cells activated without this cytokine did not become killer cells (FIG. 6A). We also considered the possibility that these CD8regs could convert to killer cells following sensitization with allogeneic dendritic cells. However, even after this procedure cytotoxic activity was negligible (FIG. 6B).

### Example 5

### Expression of TNFR2 and PD-L1 by CD8 Tregs is Essential for their Generation

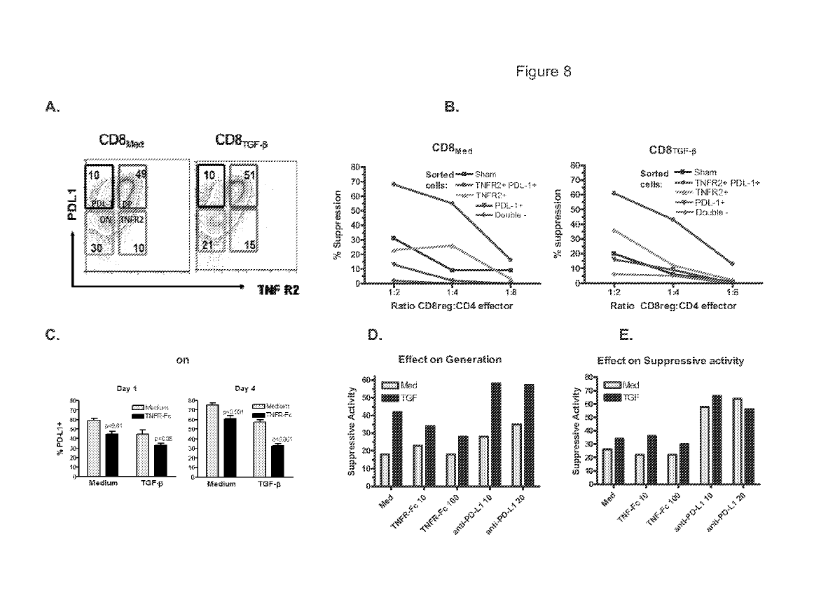

As indicated above, CD8+ cells stimulated with anti-CD3/28 beads rapidly expressed both TNFR2 and PD-L1. Unlike the rapid expression of Foxp3, however, this was not dependent on TGF-β (See FIG. 3A). To determine the significance of this finding, we deleted CD8 cells expressing these receptors by cell sorting after they were generated and assessed the suppressive activity of the remaining cells. Since, most cells expressed TNFR2 and PD-L1 by day 4 (result not shown), we sorted CD8+ cells bearing these receptors after 2 days of culture. Their FACS profile is shown in FIG. 8A. In three separate experiments we observed that TNFR2 PD-L1 double positive cells exhibited much stronger suppressive activity than control sham sorted cells (See FIG. 8B). Either TNFR2 or PD-L1 single positive cells had modest activity, but the double negative cells had none. The results were similar whether or not the CD8+ cells were conditioned with TGF-β.

We next looked for a relationship between TNFR2 and PD-L1. First, we obtained evidence that TNF upregulated PD-L1 expression. As shown in FIG. 8C, blocking TNF with soluble TNF receptors (TNFR-Fc) significantly decreased PD-L1 expression. Similarly, blocking TNF in the generation TGF-β induced CD8regs resulted in a dose-related decrease in suppressive function (See FIG. 8D). Thus, inhibition of PD-L1 expression was associated with decreased suppressive activity by TGF-β induced CD8regs. Surprisingly, anti-PD-L1 had the opposite effect and further enhanced the suppressive activity of TGF-β induced CD8regs. When added to the suppressor cell assay anti-PD-L1 also doubled activity the activity of CD8regs induced with or without TGF-β. Thus, both agents modified the suppressive effects of the CD8regs generated with anti-PD-L1 acting as an agonist instead of its usual role of an antagonist (L. M. Francisco et al., PD-L1 regulates the development, maintenance, and function of induced regulatory T cells. The Journal of experimental medicine 206, 3015 (Dec. 21, 2009)). These results suggest a role for PD-L1 in the suppressive mechanism of these CD8regs.

The above finding that non-cytotoxic, polyclonal human CD8regs generated ex-vivo with anti-CD3/28 beads, IL-2 and TGF-β preferentially targeted allogeneic T effector cells, and had in vivo at least a 2 fold greater protective effect than CD4regs has immediate clinical relevance. To date, the functional properties of natural and induced human CD8 subsets have been assessed using in vitro suppressor assays (S. D. Koch et al., Alloantigen-induced regulatory CD8+CD103+ T cells. Hum Immunol 69, 737 (November, 2008), C. Konya et al., Treating autoimmune disease by targeting CD8(+) T suppressor cells. Expert Opin Biol Ther 9, 951 (August, 2009)). However, since these methods may not correlate well with protective effects in vivo, we and others have used a human anti-mouse GVHD to assess human Treg activity. These studies have indicated that both expanded endogenous CD4regs and those induced ex-vivo can double the survival of the mice (L. Lu, et al., Characterization of protective human CD4CD25 FOXP3 regulatory T cells generated with IL-2, TGF-beta and retinoic acid. PloS one 5, e15150 (2010); R. G. Carroll et al., Distinct effects of IL-18 on the engraftment and function of human effector CD8 T cells and regulatory T cells. PloS one 3, e3289 (2008); K. L. Hippen et al., Generation and large-scale expansion of human inducible regulatory T cells that suppress graft-versus-host disease. Am J Transplant 11, 1148 (June, 2011); K. L. Hippen et al., Massive ex vivo expansion of human natural regulatory T cells (T(regs)) with minimal loss of in vivo functional activity. Sci Transl Med 3, 83ra41 (May 18, 2011); S. Amarnath et al., Regulatory T cells and human myeloid dendritic cells promote tolerance via programmed death ligand-1. PLoS Biol 8, e1000302 (February, 2010)) (See Table 1). Here we report that the polyclonal CD8regs protective effects were at least 2 fold greater than CD4regs, and these CD8regs preferentially targeted allogeneic T effector cells (FIG. 1).

The principal reason why CD8regs had greater protective effects than CD4regs may relate to the continuous stimulation required for the fitness of Tregs (K. S. Tung et al., Regulatory T-cell, endogenous antigen and neonatal environment in the prevention and induction of autoimmune disease. Immunol Rev 182, 135 (August, 2001)). Human CD4 and CD8 cells recognize differently murine MHC determinants. Human CD4+ can only respond to murine MHC gene products after they have been processed by human antigen-presenting cells (P. J. Lucas, et al., The human antimurine xenogeneic cytotoxic response. I. Dependence on responder antigen-presenting cells. Journal of immunology 144, 4548 (Jun. 15, 1990)). Since it is unlikely that human APC will remain viable in mice, the CD4regs will not receive the antigen stimulation they require to remain fit. By contrast, human CD8regs will be continuously stimulated because they can recognize polymorphic MHC I gene products expressed by mouse cells (R. E. Gress et al., Fine specificity of xenogeneic antigen recognition by human T cells. Transplantation 48, 93 (July, 1989)). Therefore, their protective activity of CD8regs should be more persistent.

The present CD8regs have a characteristic phenotype. Besides markers associated with Tregs such as Foxp3, CD25, CD122, CTLA-4, and TNFR2, these Tregs expressed the negative co-stimulator receptors PD-L1, PD-1, and Tim3. These negative co-stimulatory receptors are characteristically expressed by “exhausted” CD8+ cells following certain viral infections (E. J. Wherry, T cell exhaustion. Nature immunology 12, 492 (June, 2011)). However, while IL-2 receptors become dim on exhausted CD8+ cells (E. J. Wherry, T cell exhaustion. Nature immunology 12, 492 (June, 2011)), the CD8regs described here are CD25^{bright} and TGF-β enhanced CD122 expression. The autoantibody-suppressing CD8regs that appear in lupus patients following autologous stem cell transplantation have a phenotype quite similar to the CD8regs we have induced ex-vivo. They express Foxp3, PD-1, PD-L1, CTLA-4 and CD103 (L. Zhang et al., Regulatory T cell (Treg) subsets return in patients with refractory lupus following stem cell transplantation, and TGF-beta-producing CD8+ Treg cells are associated with immunological remission of lupus. Journal of immunology 183, 6346 (Nov. 15, 2009)). It is likely that the similar profile of negative co-stimulatory receptors expressed by CD8regs induced with and without TGF-β relates to their similar protective effects, and that one or more of these receptors these cells blocks killer cell differentiation.

The present study suggests that both IL-2 and TGF-β are needed for the generation of clinically applicable human CD8regs. This is consistent with the essential role of these cytokines in the generation and maintenance of CD4+ Foxp3+ Tregs (G. C. Furtado et al., Interleukin 2 signaling is required for CD4(+) regulatory T cell function. The Journal of experimental medicine 196, 851 (Sep. 16, 2002), D. A. Horwitz et al., Critical role of IL-2 and TGF-beta in generation, function and stabilization of Foxp3(+)CD4(+) Treg. European journal of immunology 38, 912 (April, 2008)). As with human CD4regs, TGF-β enhanced Foxp3 expression by CD8+ cells. The percentage of Foxp3+ cells expressed by the present CD8regs induced with anti-CD3/28 beads was markedly higher than reported by others Those who have used SEB or anti-CD3±anti-CD28 have observed <30% of human CD8+ Foxp3+ cells even with TGF-β (M. Mahic et al., Generation of highly suppressive adaptive CD8(+)CD25(+)FOXP3(+) regulatory T cells by continuous antigen stimulation. European journal of immunology 38, 640 (March, 2008); K. Siegmund et al., Unique phenotype of human tonsillar and in vitro-induced FOXP3+CD8+ T cells. Journal of immunology 182, 2124 (Feb. 15, 2009); V. Ablamunits et al., Acquisition of regulatory function by human CD8(+) T cells treated with anti-CD3 antibody requires TNF. European journal of immunology 40, 2891 (October, 2010)). Our range was 45 to 65% which we believe is due to the combination of anti-CD3 and anti-CD28 immobilized on beads.

Since activated human T cells can transiently express Foxp3 (M. A. Gavin et al., Single-cell analysis of normal and FOXP3-mutant human T cells: FOXP3 expression without regulatory T cell development. Proc Natl Aced Sci USA 103, 6659 (Apr. 25, 2006)), it has not been established whether this transcription factor has the same essential role in generating human CD8regs that it presumably has in mice. In this study, Foxp3 expressed by CD8+ cells was not stable and required exogenous IL-2 to maintain expression. Secondly, Foxp3 expression did not correlate with suppressive activity in vitro or in vivo. Thirdly, we observed that anti-PD-L1 inhibited Foxp3 expression (result not shown) but, surprisingly increased the CD8reg suppressive activity in vitro. Fourthly, others have reported TCR stimulated mouse CD8+ cells that express Foxp3 lack suppressive activity (C. T. Mayer et al., CD8+ Foxp3+ T cells share developmental and phenotypic features with classical CD4+ Foxp3+ regulatory T cells but lack potent suppressive activity. European journal of immunology 41, 716 (March, 2011)). Finally, using GFP to sort mouse Foxp3+ cells, we have observed that both TGF-β induced Foxp3+ and Foxp3− CD8 cells have equivalent protective activity in vivo (L. X. Ya et al., CD4+ Foxp3+CD103+ Regulatory Cells Generated ex-vivo with TGF-beta suppress autoimmunity through IL-10 dependent Mechanisms. Arthritis and rheumatism 64, S1057 (2012)). It is likely, therefore, that the protective effects observed were mediated by both Foxp3+ and Foxp3− cells. However, we cannot exclude the possibility that the human CD8+ Foxp3+ cells accounted for most of the suppressive activity observed.

TGF-β had other important effects on the human CD8+ cells besides enhancing Foxp3 expression. First, TGF-β induced CD8 cells to express CD103. Another group has also reported this finding, but they described alloantigen-induced CD8+CD103+CD28− cells that were predominantly antigen-specific (S. D. Koch et al., Alloantigen-induced regulatory CD8+CD103+ T cells. Hum Immunol 69, 737 (November, 2008)). CD8+CD103 Tregs may traffic to skin and mucous membranes (S. E. Jenkinson et al., The alphaE(CD103)beta7 integrin interacts with oral and skin keratinocytes in an E-cadherin-independent manner*. Immunology 132, 188 (February, 2011)). Although CD8+CD28− cells possess suppressive activity (N. Suciu-Foca et al., Generation and function of antigen-specific suppressor and regulatory T cells. Transpl Immunol 11, 235 (July-September, 2003)) and can comprise 1/3 of isolated CD8+ cells, following anti-CD3/28 stimulation almost all of the cells harvested are CD28+ (FIG. 3). Second, TGF-β was required for the maintenance of function in vitro, and blocking TGF-β signaling in vivo diminished the protective effect of TGF-β conditioned CD8regs. The increased stability of these CD8regs may endow them to have even more protective function in established disease than in disease prevention (M. O. Li et al., Transforming growth factor-beta controls development, homeostasis, and tolerance of T cells by regulatory T cell-dependent and -independent mechanisms. Immunity 25, 455 (September, 2006)).

Our depletion studies strongly correlating TNFR2 with suppressive activity indicates that this receptor has the same important functional role for CD8regs as it does for both murine and human CD4regs (X. Chen et al., Co-expression of TNFR2 and CD25 identifies more of the functional CD4+FOXP3+ regulatory T cells in human peripheral blood. European journal of immunology 40, 1099 (April, 2010)). Human thymic CD8+ Foxp3+ Tregs also express TNFR2, and similar to nCD4regs are anergic in vitro and do not produce cytokines (L. Cosmi et al., Human CD8+CD25+ thymocytes share phenotypic and functional features with CD4+CD25+ regulatory thymocytes. Blood 102, 4107 (Dec. 1, 2003)). The induced CD8regs described here are unlike their thymic counterparts in that they produce IL-2 and TNF, and proliferate in response to TCR stimulation in vitro. Others have also observed TNFR2 on CD8regs induced with anti-CD3 (V. Ablamunits et al., Acquisition of regulatory function by human CD8(+) T cells treated with anti-CD3 antibody requires TNF. European journal of immunology 40, 2891 (October, 2010)). Interestingly, the suppressive activity observed correlated better with TNFR2 than Foxp3. The TNF signaling through TNFR2 that generates CD8regs, therefore, may serve to balance the well-known proinflammatory effects of this cytokine. The continuous alloantigen stimulation when these Tregs are in foreign hosts may produce IL-2 and TNF that supports these CD8regs.

The rapid induction of both TNFR2 and PD-L1 on CD8 cells has not been reported previously. Both PD-1 and PD-L1 are instrumental in the maintenance of peripheral tolerance (L. M. Francisco et al., PD-L1 regulates the development, maintenance, and function of induced regulatory T cells. The Journal of experimental medicine 206, 3015 (Dec. 21, 2009)). Here TGF-β enhanced PD-1 expression by anti-CD3/28 stimulated CD8+ cells, and our evidence that TNF enhanced PD-L1 expression on CD8regs is a novel observation. Recently, others have reported that blocking TNF decreased PD-L1 expression by monocytes (N. Ou et al., TNF-alpha and TGF-beta Counter-Regulate PD-L1 Expression on Monocytes in Systemic Lupus Erythematosus. Sci Rep 2, 295 (2012)). Since soluble TNFR2 blocked both upregulation of PD-L1 and had inhibitory effects on the generation of suppressive activity, it is possible that these two observations are related. Our studies with anti-PD-L1 support this possibility. Although blocking PD-L1 expressed by antigen-presenting cells with the clone MINI used in this study antagonized the negative co-stimulatory effects of this receptor (L. M. Francisco et al., PD-L1 regulates the development, maintenance, and function of induced regulatory T cells. The Journal of experimental medicine 206, 3015 (Dec. 21, 2009)), this anti-PD-L1 antibody had agonist effects on our CD8regs. Including this mAb in the generation of CD8regs enhanced the suppressive effects of TGF-β induced CD8regs, and adding this antibody to the suppressor assay markedly enhanced suppressive activity. Thus, PD-L1 signaling may be involved in the mechanism of action of these CD8regs. Previously others have reported that overexpressing PD-L1 in Th1 cells converted these cells to CD4regs (S. Amarnath et al., The PDL1-PD1 axis converts human TH1 cells into regulatory T cells. Sci Transl Med 3, 111ra120 (Nov. 30, 2011)).

Although CD8+ cells can generally recognize and kill allogeneic target cells, we found no evidence that the CD8regs generated in this study had significant cytotoxic activity. While it is well known that TGF-β inhibits the development of killer cells (M. O. Li et al., Transforming growth factor-beta controls development, homeostasis, and tolerance of T cells by regulatory T cell-dependent and -independent mechanisms. Immunity 25, 455 (September, 2006)), and did down regulate granzyme expression in this study, CD8regs induced without TGF-β also lacked cytotoxic effects. This is consistent with the results of others who have induced polyclonal CD8regs ex-vivo (M. Mahic et al., Generation of highly suppressive adaptive CD8(+)CD25(+)FOXP3(+) regulatory T cells by continuous antigen stimulation. European journal of immunology 38, 640 (March, 2008); K. Siegmund et al., Unique phenotype of human tonsillar and in vitro-induced FOXP3+CD8+ T cells. Journal of immunology 182, 2124 (Feb. 15, 2009); V. Ablamunits et al., Acquisition of regulatory function by human CD8(+) T cells treated with anti-CD3 antibody requires TNF. European journal of immunology 40, 2891 (October, 2010); J. A. Kapp et al., TCR transgenic CD8+ T cells activated in the presence of TGFbeta express FoxP3 and mediate linked suppression of primary immune responses and cardiac allograft rejection. International immunology 18, 1549 (November, 2006); M. J. Smyth et al., Regulation of lymphokine-activated killer activity and pore-forming protein gene expression in human peripheral blood CD8+ T lymphocytes. Inhibition by transforming growth factor-beta. Journal of immunology 146, 3289 (May 15, 1991)). Specifically, one group reported anti-CD3 induced CD8regs that expressed TNFR2 that also had a non-cytotoxic mechanism of action (V. Ablamunits, K. C. Herold, Generation and function of human regulatory CD8+ T cells induced by a humanized OKT3 monoclonal antibody hOKT3gamma1 (Ala-Ala). Hum Immunol 69, 732 (November, 2008)).

Because of the observed plasticity of Tregs (M. Miyara et al., J Allergy Clin Immunol 123, 749 (April, 2009)), we also considered the possibility that non-cytotoxic CD8regs could become killer cells following exposure to allogeneic cells. Here again, cytotoxic effects were not observed, possibly because of one or more of the negative co-stimulatory receptors these CD8 cells express. The in vivo protective activity of TGF-β induced CD8regs was IL-10 dependent, and partially TGF-β dependent. IL-10 has an important role in the mechanism of natural and induced human CD8reg subsets (M. Rifa'i et al., CD8+CD122+ regulatory T cells recognize activated T cells via conventional MHC class I-alphabetaTCR interaction and become IL-10-producing active regulatory cells. International immunology 20, 937 (July, 2008); M. Gilliet, Y. J. Liu, Generation of human CD8 T regulatory cells by CD40 ligand-activated plasmacytoid dendritic cells. The Journal of experimental medicine 195, 695 (Mar. 18, 2002)). However, we cannot exclude the possibility that this blockage could also have effects on the effector T cells. TGF-β has previously been reported to have a significant role on CD8regs (J. A. Kapp, R. P. Bucy, CD8+ suppressor T cells resurrected. Hum Immunol 69, 715 (November, 2008)). As stated above, donor CD8+ Foxp3+ Tregs arise spontaneously following transplantation of MHC mismatched lymphoid cells and are not rejected (N. Sawamukai et al., Cell-autonomous role of TGFbeta and IL-2 receptors in CD4+ and CD8+ inducible regulatory T-cell generation during GVHD. Blood 119, 5575 (Jun. 7, 2012); R. J. Robb et al., Identification and expansion of highly suppressive CD8(+)FoxP3(+) regulatory T cells after experimental allogeneic bone marrow transplantation. Blood 119, 5898 (Jun. 14, 2012); A. J. Beres et al., CD8+ Foxp3+ regulatory T cells are induced during graft-versus-host disease and mitigate disease severity. J Immunol 189, 464 (Jul. 1, 2012)). One group reported that these CD8regs were even more potent than CD4regs (R. J. Robb et al., Identification and expansion of highly suppressive CD8(+)FoxP3(+) regulatory T cells after experimental allogeneic bone marrow transplantation. Blood 119, 5898 (Jun. 14, 2012)).

In this study TGF-β induced CD8regs expressed CD103. Interestingly, CD8+ cells are abundant in the deciduas of pregnant women and many are activated cells that express CD28 and CD103 (T. Tilburgs et al., F. H. Claas, Decidual CD8+CD28− T cells express CD103 but not perforin. Hum Immunol 70, 96 (February, 2009); L. Shao et al., Activation of CD8+ regulatory T cells by human placental trophoblasts. Journal of immunology 174, 7539 (Jun. 15, 2005)). TGF-β producing trophoblasts induce CD8+ CD28+ cells to express CD103 and the proliferating fraction develops suppressive activity (L. Shao et al., Activation of CD8+ regulatory T cells by human placental trophoblasts. Journal of immunology 174, 7539 (Jun. 15, 2005)). These cells do not express perforin and also lack cytotoxic activity. These two groups suggest that the principal function these CD8regs is to maintain maternal/fetal tolerance. The fact that our Tregs preferentially target allogeneic cells, and that donor CD8+ Foxp3+ Tregs spontaneously appear in graft versus host disease (N. Sawamukai et al., Cell-autonomous role of TGFbeta and IL-2 receptors in CD4+ and CD8+ inducible regulatory T-cell generation during GVHD. Blood 119, 5575 (Jun. 7, 2012); R. J. Robb et al., Identification and expansion of highly suppressive CD8(+)FoxP3(+) regulatory T cells after experimental allogeneic bone marrow transplantation. Blood 119, 5898 (Jun. 14, 2012); A. J. Beres et al., CD8+ Foxp3+ regulatory T cells are induced during graft-versus-host disease and mitigate disease severity. J Immunol 189, 464 (Jul. 1, 2012)) supports the hypothesis that these CD8regs are important in maintaining tolerance.

There are limitations to this study. Because GVHD in the xenograft system is CD4 dependent (J. Wilson et al., Antibody to the dendritic cell surface activation antigen CD83 prevents acute graft-versus-host disease. J Exp Med 206, 387 (Feb. 16, 2009)) and their activation is dependent upon cross presentation by the human antigen-presenting cells transferred, this is not a good model to study human class I restricted responses. We have used this model in humanized mice because in vitro suppressive assays may not reflect protective activity of Tregs in vivo. There are also limitations of mouse models in studying human immune regulation. We and others have previously observed that although IL-2 and TGF-β induce mouse CD4 cells to become Tregs that are protective in vivo (D. A. Horwitz et al., Natural and TGF-beta-induced Foxp3(+)CD4(+) CD25(+) regulatory T cells are not mirror images of each other. Trends in immunology 29, 429 (September, 2008)), another agent such as retinoic acid must be added to this combination to rapidly induce human naïve CD4+ cells to have similar activity (L. Lu, et al., Characterization of protective human CD4CD25 FOXP3 regulatory T cells generated with IL-2, TGF-beta and retinoic acid. PloS one 5, e15150 (2010)). In the present experiments, the preferential effect of human induced CD8regs on allogeneic T cells is not observed in mouse models.

The finding that the polyclonal CD8regs generated with anti-CD3/28 beads preferentially target allogeneic cells raise the possibility that Tregs from an unrelated donor could be used for T cell immunotherapy. Allogeneic cells transferred from one unrelated individual to another should be rejected. However, since the suppressive activity of the transferred CD8regs will be continuously strengthened by contact with the recipient's cells, they may prevent the recipient from mounting an immune response against them. If non-cytotoxic, appropriately typed allogeneic CD8regs can be administered without being rejected and maintain their protective activity without serious adverse side effects, they have the potential to revolutionize our treatment of autoimmune diseases, graft-versus-host disease, and allograft rejection.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

### Example 6

Immunodeficient NOD SCID IL-2 receptor common gamma chain deficient mice are injected IP with the peripheral blood mononuclear cells obtained from a lupus patient with a high titer of anti-DNA antibodies. These mice are unable to reject human tissue and within 2 weeks, human T cells are detected in mouse blood samples, and human IgG and anti-DNA antibodies become detectable in the mouse serum. The mice then receives 5 million CD8+ regulatory T cells generated ex-vivo with a T cell mitogen, IL-2, and TGF-β from peripheral blood mononuclear cells obtained from an unrelated subject. Over 4 weeks both IgG levels and anti-DNA antibodies decrease markedly and by 8 weeks disappear completely. During this time, the mice gain weight and appear healthy.

### Example 7

A 22 year old female graduate student presents to a rheumatologist with rash, alopecia, arthritis, chest pain with inspiration, mouth ulcers, loss of appetite, and easy fatigability for two months. The pain and swelling in her hands and wrists are especially severe in the morning. The laboratory exam reveals a modest anemia, hematuria, proteinuria, a strongly positive anti-nuclear antibody, decreased serum complement, and strongly positive anti-double stranded DNA antibody test. Her blood urea nitrogen and serum creatinine are normal. A Coombs test is positive. A diagnosis of systemic lupus erythematosus is made and she is instructed to take hydroxychloroquine 200 mg twice a day, and prednisone 60 mg for 3 months. Her symptoms improve rapidly, the rash diminishes, arthritis disappears and her energy level increases. However, these symptoms return when the prednisone is reduced to 20 mg a day. At that time, a 24 hour urine shows 4 grams of protein. A kidney biopsy reveals a class IV proliferative nephritis consistent with lupus. From blood donated by a haploidentical sister regulatory CD8+ T cells are generated ex-vivo with a T cell mitogen and co-stimulatory anti-CD28 immobilized together, IL-2, and TGF-β. The CD8+ cells are expanded with IL-2. The patient then receives three IV infusions of regulatory T cells generated from CD8+ peripheral blood mononuclear cells in the next three weeks. Again all symptoms improve. Within the next two months her anemia is resolved, the serum complement levels return to normal and the anti-DNA antibodies become undetectable. Red blood cells are no longer detectable in her urine, and the proteinuria decreases to trace. The Coombs test becomes negative. The prednisone dose is reduced to 5 mg and she is able to resume fully her academic activities.

What is claimed is:

1. A method comprising administering suppressor cells to a patient, wherein said suppressor cells are allogeneic to said patient and wherein said administrating is for other than the treatment or prevention of graft versus host disease.

2. A method comprising administering suppressor cells to a patient, wherein said suppressor cells are allogeneic to said patient and wherein said administrating is for the treatment or prevention of an immune disorder.

3. The method of claim 3 wherein said immune disorder is an autoimmune disorder.

4. A method comprising administering suppressor cells to a patient, wherein said suppressor cells are allogeneic to said patient and wherein said administrating is for the treatment or prevention of organ transplant rejection.

5. The method of claims 1 through 4 wherein said suppressor cells are generated by a method comprising contacting blood or a component of blood comprising CD8+ cells with a suppressor-inducing composition to activate said CD8⁺ cells to become suppressor cells.

6. The method of claim 5 wherein said suppressor-inducing composition comprises anti-CD-28, anti-CD3 and IL-2

7. The method of claims 1 through 4 wherein said suppressor cells are CD8⁺ cells.

8. The method of claims 1-4 wherein said suppressor cells are resistant to rejection by said patient's immune system.

9. The method of claims 1-4 wherein said suppressor cells inhibit allogeneic immune cells in said patient and wherein said suppressor cells have stronger activity against allogeneic cells than autologous cells.

10. The method of claims 1-4 wherein said suppressor cells are generated from a donor who matches at least one set of MHC antigens but does not match all MHC antigens in said patient.

11. The method of claims 1-4 wherein said allogeneic suppressor cells are administered to said patient without other immunosuppressive therapies.

12. The method of claims 1-3 wherein said suppressor cells are further expanded by culturing in the presence of MHC antigens from said patient prior to treating said patient.

13. The method of claim 4 wherein said suppressor cells are further expanded by culturing in the presence of MHC antigens from said patient and the donor of said organ prior to treating said patient.

14. A method to treat or prevent graft versus host disease in a patient receiving donor bone marrow cells comprising selecting a third party CD8⁺ suppressor cell that is partially matched to the MHC antigens of the patient and the MHC antigens of the donor, expanding said third party CD8⁺ suppressor cells by culturing in the presence of the MHC antigens from the donor and optionally the MHC antigens from the patient, and the administering said expended third party CD8⁺ suppressor cells and said donor cells to said patient.

15. The method of claim 14 wherein said expanded CD8⁺ suppressor cells are administered to said patient before said donor cells.

16. The method of claim 14 wherein said expanded CD8⁺ suppressor cells and said donor cells are simultaneously administered to said patient.

17. A method to treat or prevent tissue or organ rejection in a patient receiving a tissue or organ transplant from a donor comprising selecting a third party CD8⁺ suppressor cell that is partially matched to the MHC antigens of the patient and the MHC antigens of the donor, expanding said third party CD8⁺ suppressor cells by culturing in the presence of the MHC antigens from the donor and the MHC antigens from the patient, and administering said expended third party CD8 suppressor cells and said donor tissue or organ to said patient.

18. The method of claim 17 wherein said expanded CD8⁺ suppressor cells are administered to said patient before said donor cells.

19. The method of claim 17 wherein said expanded CD8⁺ suppressor cells and said donor tissue or organ are simultaneously administered to said patient.

20. A method for generating suppressor cells, said method comprising:
(a) activating CD8⁺ cells with a suppressor-inducing composition, and
(b) expanding said activated CD8⁺ cells in the presence of at least two MHC antigens which are different from each other and from the MHC of said CD8⁺ cells.

21. The method of claim 20 wherein said suppressor-inducing composition comprises anti-CD3, anti-CD28 and IL-2

22. The method of claim 20 wherein said CD8⁺ cells are further treated with TGF-β.

23. The method of claim 20 wherein said MHC antigens are from peripheral blood mononuclear cells that are allogeneic to said CD8⁺ cells.

24. The method of claim 23 wherein one of said MHC antigens is from a patient to be treated with said suppressor cells.

25. The method of claim 24 wherein the other of said MHC antigens is from a donor of cells, tissue or organs to be transplanted to said patient.

25. The method of claim 20 wherein said activating and said expanding are carried out simultaneously.

26. A composition comprising CD8⁺ suppressor cells generated by activating naïve CD8⁺ cells by treating blood, a component of blood comprising naïve CD8+ cells or isolated naïve CD8⁺ cells with a suppressor-inducing composition and expanding said activated CD8⁺ cells by culturing in the presence of at least first and second different MHC antigens which are allogeneic to the MHC antigens of said CD8⁺ cells.

27. The composition of claim 26 wherein said suppressor-inducing composition comprises anti-CD3, anti-CD28 and IL-2

28. The composition of claim 26 wherein said first different MHC antigens are from a cell, tissue or organ donor and said second different MHC antigens are from a recipient of said cell, tissue or organ.

29. The composition of claim 28 wherein said CD8⁺ suppressor cells inhibit allogeneic cells comprising said first and second MHC antigens when administered to said recipient.

30. The composition of claim 26 wherein said wherein said composition is frozen.

31. A suppressor cell bank comprising a collection of containers, wherein each container comprises CD8⁺ suppressor cells and wherein at least two of said containers contain CD8+ suppressor cells that have different MHC antigens.

32. The suppressor cell bank of claim 31 comprising 10 or more CD8⁺ suppressor cells that have different MHC antigens.

33. The suppressor cell bank of claim 31 wherein said suppressor cells are frozen.
